(19) United States
(12) Patent Application Publication
(10) Pub. No.: US 2026/0108214 A1
(43) Pub. Date: Apr. 23, 2026

(54) CANCER PATIENT MANAGEMENT SYSTEM

(71) Applicant: Gregory MCMAHON, Newtown Square, PA (US)

(72) Inventor: Gregory MCMAHON, Newtown Square, PA (US)

(21) Appl. No.: 19/360,393

(22) Filed: Oct. 16, 2025

Related U.S. Application Data
(60) Provisional application No. 63/708,838, filed on Oct. 18, 2024.

(51) Int. Cl. A61B 5/00

(52) U.S. Cl. CPC A61B 5/7267; A61B 5/4836; A61B 5/7275; A61B 5/742; A61B 5/7475

(57) ABSTRACT

A patient management system and method for providing a risk profile and care plan to a patient and provider. The patient management system includes a risk analysis module configured to convert unstructured data to structured data and includes a data acquisition module for collecting and transmitting unstructured data and a classifier module for analyzing unstructured data and converting the unstructured data into structured data. The data acquisition module receives the unstructured data set, and the classifier module converts the unstructured data to a structured data set. A provider interface system is in communication with the classifier module and is configured to collect a plurality of structured data sets, analyze the collected structured data sets and output a risk profile/group to a provider.

### RELATED APPLICATIONS

This application claims priority to U.S. Provisional Patent Application 63/708,838, filed Oct. 18, 2024, entitled, “CANCER PATIENT MANAGEMENT SYSTEM”, which is incorporated by reference herein in its entirety.

### FIELD OF THE INVENTION

The present disclosure is generally directed to cancer patient management.

### BACKGROUND OF THE INVENTION

Assessing individual cancer risk in groups of patient groups is a difficult and time-consuming process requiring significant medical knowledge and skill. Each patient, including their tests results, must be evaluated individually by a physician to determine the individual patient's risk and determine proper treatment. For example, no program exists to identify an individual male's prostate cancer risk group. Similarly, no program exists to identify a patient's risk group with non-muscle invasive bladder cancer or if their bladder cancer is BCG unresponsive. Proper identification enables improved patient outcomes as well as the potential for increased revenue.

Men with high and very high-risk prostate have an increased risk of having metastatic disease at the time of diagnosis as well as an increased risk of cancer reoccurrence following a primary treatment. Therefore, several advanced forms of diagnostic imaging, genetic testing, and treatment intensification are available to men with high or very high-risk prostate cancer.

Diagnostics, treatments, and clinical trials for bladder cancer are often dependent on the patient's risk group. For instance, the National Comprehensive Cancer Network and American Urology Association have set forth criteria to categorize bladder cancer risk groups. In patients with non-muscle invasive bladder cancer higher risk groups are associated with more severe disease and odds of developing progression or metastasis. Bladder cancer is also one of the most expensive cancers to treat over a patient's lifetime in part due to the chance of reoccurrence. Proper risk identification may allow for treatment intensification and thus lowering of the cost to the healthcare system.

In many clinical settings the information needed to properly risk stratify a patient with cancer comes from numerous data sources some of which are unstructured, making analysis difficult. For instance, in some clinical settings a practice may receive biopsy information from several laboratories or radiology reports from several radiology departments. The reports from each entity are structured differently, making interpretation cumbersome. The inability to easily review and analyze this information can have a negative impact both patient care and a medical practices revenue stream.

For instance, in men with very high-risk prostate cancer electing for radiation as a primary treatment the addition of androgen deprivation (medication) and a novel hormone therapy (medication—Abiraterone) has been shown to significantly improve metastasis free and overall survival. Additionally, many urology or medical oncology practices have ownership interest in diagnostic and therapeutics available to these patients. Proper risk group identification increases a practice's revenue.

Using bladder cancer as an example, in patients with high and very high-risk non-muscle invasive bladder cancer or BCG-unresponsive disease several novel therapies and numerous clinical trials are available. These treatments have the potential to stop the disease from progressing which leads to metastatic disease or the need for a radical cystectomy (bladder removal). Likewise, to prostate cancer, efficient identification of these patients increases a practices revenue.

What is needed is a system for identifying and quantifying individual cancer risk to determine potential treatments that do not suffer from the drawbacks in the prior art. Other features and advantages will be made apparent from the present specification. The teachings disclosed extend to those embodiments that fall within the scope of the claims, regardless of whether they accomplish one or more of the aforementioned needs.

### SUMMARY OF THE INVENTION

Embodiments of the present disclosure include a cancer patient management system that allows partial quantification of input data to identify an individual cancer risk group. An embodiment of the present disclosure includes a patient management system. The patient management system includes a risk analysis module configured to convert unstructured data to structured data and includes a data acquisition module for collecting and transmitting unstructured data and a classifier module for analyzing unstructured data and converting the unstructured data into structured data. The data acquisition module receives the unstructured data set, and the classifier module converts the unstructured data to a structured data set. A provider interface system is in communication with the classifier module and is configured to collect a plurality of structured data sets, analyze the collected structured data sets and output a risk profile to a provider.

Another embodiment of the present disclosure includes a non-transitory machine-readable storage medium storing one or more sequences of instructions for generation of a risk profile for a cancer patient, which when executed by one or more processors, cause a risk analysis module to convert unstructured data to structure data. The risk analysis module includes a data acquisition module to receive an unstructured data set and a classifier module to analyze the unstructured data set and convert the unstructured data into a structured data set. A provider interface system in communication with the classifier module is caused to collect a plurality of structured data sets, analyze the collected structured data sets and output a risk profile to a provider.

Another embodiment of the present disclosure includes a method for providing a risk profile and care plan to a patient. The method includes providing unstructured data from a testing system, converting unstructured data to structured data with a risk analysis module in communication with the testing system, aggregating structured data with a provider interface system to determine a risk score, and providing the risk score to a provider with the provider interface system.

Other features and advantages of the present invention will be apparent from the following more detailed description of the preferred embodiment, taken in conjunction with the accompanying drawings which illustrate, by way of example, the principles of the invention.

### BRIEF DESCRIPTION OF DRAWINGS

FIG. 1 shows a schematic view of a patent management system according to an embodiment of the present disclosure.

FIG. 2 shows a schematic view of an information system according to an embodiment of the present disclosure.

FIG. 3 shows a schematic view of a data processing system 300 according to an embodiment of the present disclosure.

FIG. 4 shows exemplary output provided in the provider interface system according to the present disclosure.

FIG. 5 shows exemplary output provided in the provider interface system according to another embodiment of the present disclosure.

FIG. 6 shows exemplary output provided in the provider interface system according to another embodiment of the present disclosure.

FIG. 7 shows exemplary output provided in the provider interface system according to another embodiment of the present disclosure.

FIG. 8 shows exemplary analysis criteria according to an embodiment of the present disclosure.

FIG. 9 shows exemplary output based on the analysis criteria shown in FIG. 8.

FIG. 10 shows exemplary analysis criteria according to another embodiment of the present disclosure.

FIG. 11a shows exemplary output provided in the provider interface system according to another embodiment of the present disclosure.

FIG. 11b shows a highlighted portion including cell content of the exemplary output of FIG. 11a.

FIG. 12a shows exemplary output provided in the provider interface system according to another embodiment of the present disclosure.

FIG. 12b shows a highlighted portion including cell content of the exemplary output of FIG. 12a.

FIG. 13a shows exemplary output provided in the provider interface system according to another embodiment of the present disclosure.

FIG. 13b shows a highlighted portion including cell content of the exemplary output of FIG. 13a.

FIG. 14a shows output provided in the provider interface system including exemplary analysis criteria according to another embodiment of the present disclosure.

FIG. 14b shows a highlighted portion including cell content of the exemplary output of FIG. 14a.

FIG. 15 shows exemplary analysis criteria according to another embodiment of the present disclosure.

FIG. 16a shows exemplary output provided in the provider interface system according to another embodiment of the present disclosure.

FIG. 16b shows exemplary output provided in the provider interface system according to another embodiment of the present disclosure.

FIG. 16c shows exemplary output provided in the provider interface system according to another embodiment of the present disclosure.

FIG. 16d shows exemplary output provided in the provider interface system according to another embodiment of the present disclosure.

FIG. 16e shows exemplary output provided in the provider interface system according to another embodiment of the present disclosure.

FIG. 16f shows exemplary output provided in the provider interface system according to another embodiment of the present disclosure.

FIG. 16g shows exemplary output provided in the provider interface system according to another embodiment of the present disclosure.

FIG. 16h shows exemplary output provided in the provider interface system according to another embodiment of the present disclosure.

Wherever possible, the same reference numbers will be used throughout the drawings to represent the same parts.

### DETAILED DESCRIPTION OF THE INVENTION

A patient management system has been developed to provide accurate identification of cancer risk groups and further diagnostic tests and therapeutics by integrating multiple components that work together seamlessly. A testing system may conduct diagnostic tests for each patient based on pre-defined criteria stored in an electronic health record (EHR) database, generating unstructured data as output. This raw information is provided to the risk assessment module where the unstructured data is converted into structured data, such as a binary or linear format, through a process of reviewing individual data sources and assigning values to specific parameters. By converting unstructured data into structured data, the system according to the present disclosure appropriately risk stratify individual patients. This allows for increased patient care and practice revenue. In one embodiment, unstructured data is transformed into structured data by reviewing each individual data source and converting it into structured data, such as a binary (0 or 1) value or linear (1, 2, 3, 4 . . . ) value.

There are numerous advantages to the system according to embodiments of the present disclosure. From a patient care perspective, timely and correct cancer risk identification leads to improved outcomes. Specifically, the outcomes previously studied included increased metastasis-free survival (Metastasis-free survival is a surrogate end-point commonly used in prostate cancer to predict overall survival) and overall survival (overall survival refers to how long someone lives with a disease). Diagnosing a patient with high or very high-risk cancer is cumbersome due to the need to review and interpret multiple data sources and convert that data into a structured format. The system according to the present disclosure is easy to use and allows for quick and repetitive process leading to timely identification of these patients therefore allowing them to get on life prolonging therapy.

Additionally, many medical groups (independent practices, hospitals, academic centers, etc.) have their own pharmacy in which they can prescribe and administer the drugs indicated in men with high and very high-risk prostate cancer. By helping to identify these patients, the system according to the present disclosure helps to substantially increase a practices revenue stream. For example, if only 33% of men with very high-risk clinically localized prostate cancer electing for radiation are offered abiraterone currently. Said differently, two-thirds of men who qualified for treatment intensification where not being appropriately identified and offered life-prolonging therapy. Additionally, in situations in which the clinical practice has financial ownership in a pharmacy, the system according to the present disclosure leads to increased drug utilization and thus increased revenue from their pharmacy.

In practices that have higher level diagnostic capabilities, such as germline testing and prostate specific membrane antigen CT/PET imaging capabilities the system according to the present disclosure would also help to identify men who would qualify for both. For example, the system according to the present disclosure results in increased utilization of germline testing and imaging that leads to an increase in revenue for practices with ownership of these diagnostic capabilities.

### Definitions

Prostate cancer: Most common cancer impacting males and a leading cause of cancer related deaths.

Prostate cancer Risk Groups: The National Comprehensive Cancer Network (NCCN) has published risk groups. A patient is matched into a risk group based off on several diagnostic criteria (Prostate biopsy, Prostate Specific Antigen, Multiparametric MRI, etc.).

Prostate specific antigen: a blood test frequently preformed to help screen and follow a man's response to treatment for prostate cancer.

Multiparametric prostate MRI: A form of imaging (MRI) that allows for the identification of lesions in the prostate worrisome for prostate cancer. MRI's can also report if the prostate cancer has spread beyond the prostate (Extracapsular Extension).

Gleason Group: The Gleason Group is a score reported by a pathologist after reviewing a patient's prostate biopsy or prostate gland. The Gleason Group ranges from 1-5. The higher the number the more aggressive the prostate cancer is.

Gleason Score: The Gleason Score is another way a pathologist can communicate the architecture or aggressiveness of an individual's prostate cancer. For example, a Gleason score of 3+3=6 equals a Gleason Group of 1.

High Risk Prostate Cancer: The definition of High-Risk Prostrate Cancer is generally understood in the art and may change over time. For example, one suitable definition of “High-Risk” Prostate Cancer includes testing showing a patient having at least one of the following: PSA>20, extracapsular extension T3a disease (MRI), Grade Group 4 or 5 disease (prostate biopsy information).

Very High—Risk Prostate Cancer: The definition of Very High-Risk Prostrate Cancer is generally understood in the art and may change over time. For example, one suitable definition of “Very High-Risk” Prostate Cancer includes testing showing a patient having two or more high risk criteria or one or more of the following: seminal vesicle invasion T3b disease (MRI), Greater than 4 cores positive for GG4 or 5 disease (biopsy), primary Gleason score of 5 (Biopsy), PSA>40.

Bladder Cancer: A common occurring cancer impacting both men and females. The cancer occurs in the urinary bladder and can be very aggressive with a poor prognosis in advanced risk groups and stages.

Non-Muscle Invasive Bladder Cancer: A bladder cancer that invades the superficial layers of the bladder such as the mucosa (Ta) or submucosa (T1). This is evaluated and reported in the pathology report. In a majority of cases the goals of treatment with non-muscle invasive bladder cancer are to preserve the bladder (avoid a cystectomy/bladder removal).

Muscle Invasive Bladder Cancer: A bladder cancer that at least invades the muscularis propria (T2). This is evaluated and reported in the pathology report. The standard of care in patients with muscle invasive disease is to have their bladder removed.

Bladder Cancer Grade: How bladder cancer cells appear under a microscope. Grade is assigned by a pathologist and can be Low-Grade or High-Grade. This is evaluated and reported in the pathology report.

Carcinoma in-Situ: A distinct form a bladder cancer that by definition is considered High-Grade. This is evaluated and reported in the pathology report.

### Reoccurrence: Time From Last Diagnosis to Discovery of Recurrent disease.

Low-Risk Non-Muscle Invasive Bladder Cancer: A bladder cancer that has a low-risk of progressing to muscle invasive disease. The definition of Low-Risk Non-Muscle Invasive Bladder Cancer is generally understood in the art and may change over time. For example, one suitable definition of “Low-Risk” Non-Muscle Invasive Bladder Cancer includes testing showing presence of Low-grade disease AND mucosa only involvement (Ta), less than 3 cm, and solitary.

Intermediate-Risk Non-Muscle Invasive Bladder Cancer: A bladder cancer that has an intermediate-risk of progressing to muscle invasive disease. The definition of Intermediate-Risk Non-Muscle Invasive Bladder Cancer is generally understood in the art and may change over time. For example, one suitable definition of “Intermediate-Risk” Non-Muscle Invasive Bladder Cancer includes testing showing either 1) Low Grade AND Mucosa only involvement (Ta) AND greater than 3 cm or multifocal 2) Recurrence within one year of low-Risk non-muscle invasive bladder cancer 3) Submucosal involvement (T1) AND low-grade, 4) High-Grade, mucosal only involvement (Ta), and less than 3 cm.

High-Risk Non-Muscle Invasive Bladder Cancer: A bladder Cancer that has a high-risk of progressing to muscle invasive disease. The definition of High-Risk Non-Muscle Invasive Bladder Cancer is generally understood in the art and may change over time. For example, one suitable definition of “High-Risk” Non-Muscle Invasive Bladder Cancer includes testing showing either 1) Any recurrent high-grade, mucosal only involvement (Ta) 2) High-grade, mucosal only involvement, AND greater than 3 cm 3) Any Carcinoma in-situ (CIS) 4) Any High-grade AND submucosal involvement (T1).

Very High-Risk Non-Muscle Invasive Bladder Cancer: A bladder cancer that has the highest risk of progressing to muscle invasive disease. The definition of Very High-Risk Non-Muscle Invasive Bladder Cancer is generally understood in the art and may change over time. For example, one suitable definition of “Very High-Risk” Non-Muscle Invasive Bladder Cancer includes testing showing presence of any of the following: Variant Histology record of pathology, Lymphovascular invasion recorded on pathology, High-grade prostatic urethral invasion, Bacillus Calmette-Guerin (BCG) unresponsive disease.

Bacillus Calmette-Guerin (BCG): An attenuated virus commonly used as a vaccine. BCG can be instilled into the bladder to treat forms of non-muscle invasive bladder cancer to help prevent disease reoccurrence and/or progression.

Adequate BCG: The definition of Adequate BCG is generally understood in the art and may change over time. For example, one suitable definition of Adequate BCG includes a definition from the Food and Drug Administration as 5/6 induction doses AND 2/3 first maintenance doses OR 5/6 induction doses AND 2/6 second induction doses.

Bacillus Calmette-Guerin Unresponsive disease: Defined by the Food and Drug Administration as: 1) Persistent or recurrent CIS alone OR persistent or recurrent CIS with recurrent Ta/T1 disease within 12 months of adequate BCG 2) Recurrent high-grade Ta/T1 within 6 months of adequate BCG 3) High-Grade T1 at the first evaluation following BCG induction BCG.

Bladder biopsy: A procedure in which a small piece of the bladder is obtained for pathological examination.

Transurethral resection of the bladder (TURBT): A procedure in which a bladder tumor(s) is/are removed partially or completely providing both a diagnostic and therapeutic benefit.

Structured data: Data or information that is in a structured or semi-structured form, such as a numerical value, permitting comparisons across multiple structured data sets. For example, structured data may be binary or linear values that have been derived with the classifier module. The classifier module may use, for example, provided criteria from a provider or more automated methods, such as natural language processing techniques like text mining, entity recognition, and sentiment analysis to extract relevant information from clinical notes, radiology reports, pathology slides, or other sources. Data points that are easily obtained and manipulated. For example, PSA values that can easily be obtained, imported, exported, from a data source such as an electronic medical record. Example: transformation of unstructured data into binary “0” or “1” or a linear format “1, 2, 3, 4 . . . ”.

Un-structured data: Data points that are not easily obtained and manipulated, such as MRI and Prostate biopsy reports. To extract data points manual review, machine learning, Artificial Intelligence are needed to convert the unstructured data into semi-structured data which can be analyzed.

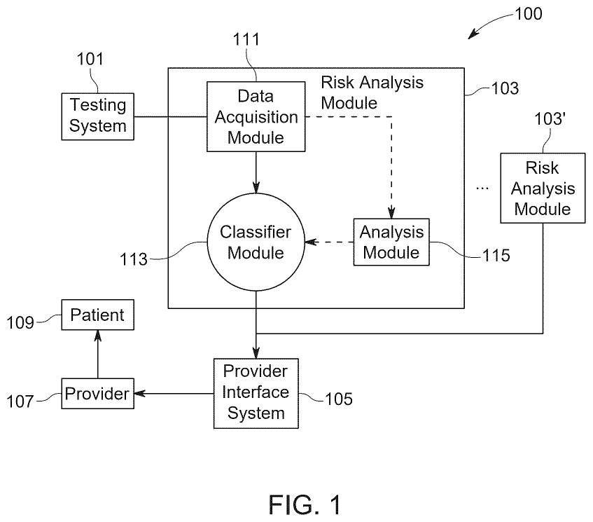

FIG. 1 shows a patient management system 100 according to an embodiment of the present disclosure. The patient management system 100 includes testing system 101, a risk analysis module 103, and a provider interface system 105. In addition, the patient management system 100 further includes a provider 107 that provides a care plan a patient 109. The risk analysis module 103 includes a data acquisition module 111 and classifier module 113. In addition, optionally and shown as a dotted line, the risk analysis module 103 may include an analysis module 115 may use advanced analysis techniques, such as text mining, entity recognition, sentiment analysis techniques to analyze acquired unstructured data to provide to the classifier module 113 to convert the analyzed unstructured data to structured data.

As shown in FIG. 1, the data acquisition module 111 receives the unstructured data set from testing system 101 and provides the unstructured data to the classifier module 113 that converts the unstructured data into structured data. Structured data, as utilized herein, means data that is in a structured or semi-structured form, such as a numerical value, permitting comparisons across multiple structured data sets. For example, structured data may be binary or linear values that have been derived with the classifier module 113 using a provided criteria or using natural language processing techniques like text mining, entity recognition, and sentiment analysis to extract relevant information from clinical notes, radiology reports, pathology slides, or other sources. For example, in the instance of prostate cancer, multiparametric imaging (MRI's) may be the testing system 101, which may be reviewed by the analysis module 115 and/or the classifier module 113 for extracapsular extension. If no extracapsular extension is reported a value of “0” is assigned as the structured data. If extracapsular extension is present a value of “1” is assigned as the structured data. Likewise, this is performed for seminal vesicle invasion/involvement “0” not involved “1” involved/invasion as the structured data, if pathology shows grade-group 4 or 5 disease “0” if not present and “1” if present as the structured data, PSA value greater than 20 “0” if the PSA is less than 20 and “1” if 20 or greater as the structured data, and if the primary Gleason score is 5, “0” if not 5 and “1” if 5 as the structured data. For linear data, the data from the testing system 101 is collected by the data acquisition module 111 and is reviewed by the classifier module 113 and/or the analysis module 115, and a “count” is entered as the structured data. For example, if a prostate biopsy has 12 samples and 6 of those samples have a grade-group of 4 or 5 disease the count would be “6” as the structured data. This linear number is entered into a dedicated field and transformed into binary data by asking is the total number of biopsy cores with grade-group 4 or 5 disease>4 “0” no “1” yes as the structured data. By converting all the unstructured data into a binary or linear format as the structured data, the created structure that can be used for analysis by the provider interface system 105.

Also shown in FIG. 1, is additional risk analysis modules 103′ which may include the data acquisition module 111, the classifier module 113 and optionally the analysis module 115, as described above. While only two risk analysis modules 103 are shown in FIG. 1, any number of risk analysis module 103′ may be utilized, as is desired for a particular type of cancer and cancer testing to develop a care plan for a patient 109.

The classifier module 113 may employ, for example, machine learning algorithms trained on large datasets of structured clinical information including demographics, medical history, laboratory results, imaging studies, pathology reports, operative notes, and other factors. These models may be adjusted based on individual patients' characteristics through active learning techniques involving human-in-the-loop validation and feedback mechanisms to ensure accuracy and reliability in patient profiling. In addition to machine learning, Optical Character Recognition (OCR) or text-based PDF extraction may be used to convert images of text into a machine-readable text.

In one embodiment, the provider interface system 105 may prioritize data collection from various sources using algorithms that assess the reliability of each input source such as EHR systems, laboratory information systems, radiology imaging platforms. Provider interface system 105 may also weigh certain data more heavily considering clinical information including demographics, medical history, laboratory results, imaging studies, pathology reports, operative notes and other relevant factors. Patient-specific factors like age, medical history, lifestyle habits, family history genetic predisposition environmental exposures may influence analysis by provider interface system 105 for accurate risk assessment through incorporation of these variables into machine learning models. Missing values or incomplete patient records may impact accuracy, but the system incorporates quality control measures such as data validation checks peer review processes and continuous monitoring performance metrics against established benchmarks.

The risk analysis module and the provider interface system 105 adhere to industry-standard storage requirements secure handling sensitive medical information, HIPAA compliance protocols encryption algorithms like AES-256 bit keys. The knowledge base is regularly updated with new evidence-based recommendations from reputable sources including but not limited to, professional societies, national guidelines, and government agencies ensuring accuracy reliability patient profiling.

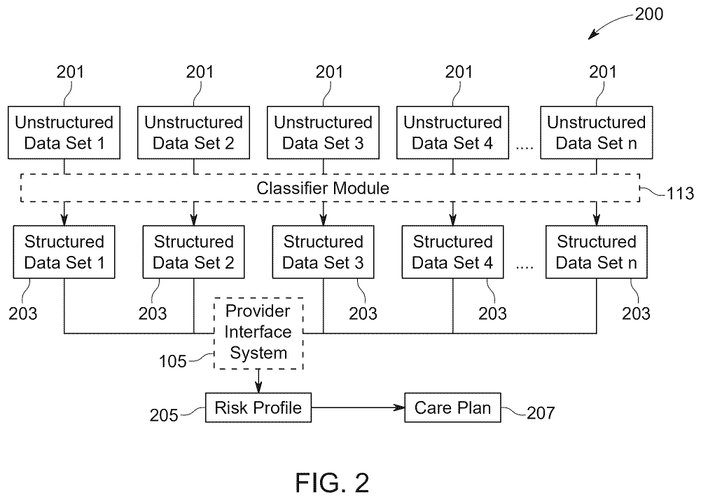

FIG. 2 shows information system 200 according to an embodiment of the present disclosure. As shown in FIG. 2, a plurality of unstructured data sets 201 are obtained, for example from testing system 101, as shown and described in FIG. 1. The unstructured data sets 201 are converted to structured data sets 203 by classifier module 113, as shown and described with respect to FIG. 1. The structured data sets 203 are provided to the provider interface system 105 and a risk profile 205 is provided. The risk profile 205 is then utilized to develop a care plan 207. The care plan 207 can be communicated by the provider 107 to patient 109.

In one embodiment the testing system 101 collects diagnostic tests for prostate cancer patients generating unstructured data sets 201 as output, such as MRI reports pathology results operative notes and other relevant information, which are then converted into structured data sets 203, which may include binary or linear numerical format with the classifier module 113. The classifier module 113 may employ reinputted thresholds or relationships or may include machine learning algorithms trained on large datasets of clinical information to predict risk profiles based on individual patient characteristics.

In another embodiment the testing system 101 conducts diagnostic tests for bladder cancer patients generating unstructured data sets 201 as output, such as pathology reports operative notes and other relevant information, which are then converted into structured data sets 203, which may include binary or linear numerical format with the classifier module 113. Like in the example of the prostate cancer testing, the classifier module 113 may employ reinputted thresholds or relationships or may include machine learning algorithms trained on large datasets of clinical information to predict risk profiles based on individual patient characteristics. In other embodiments, the patient management system 100 may be utilized for other cancers or disease. In these embodiments, the risk analysis module 103 would receive unstructured data sets from a particular test or diagnosis to form structured data sets. In addition, the provider interface system 105 would be adapted for the particular type of cancer or disease.

The present disclosure provides a useful system that may be used for accurate identification of cancer risk groups and further diagnostic tests and therapeutics in various embodiments, each tailored to specific types of cancers such as prostate or bladder cancer, allowing healthcare providers to provide an informed care plan, which includes informed decisions about treatment options.

The testing system 101 is a useful component of the patient management system that conducts diagnostic tests for cancer patients and generates unstructured data as an output. In one embodiment, this test may be performed using multiparametric magnetic resonance imaging (MRI) to assess extracapsular extension in prostate cancer cases. The MRI reports are then reviewed by the classifier module 113, which converts raw unstructured data into structured data that may be analyzed by the provider interface system 105. In another embodiment, the testing system 101 may use pathology results from a biopsy procedure as input data for risk analysis. For example, in bladder cancer cases, the data acquisition module 111 collects operative reports to determine tumor size and number of tumors. In yet another embodiment, the testing system 101 may utilize laboratory results from blood tests as input data for risk analysis. For instance, in prostate cancer cases, data acquisition module 111 reviews PSA values greater than 20 and assigns a value of “1” if present.

The data acquisition module 111 is a useful component of the patient management system that contributes to converting raw unstructured data into structured binary or linear format for analysis by the classifier module 113. The data acquisition module 111 may be configured, for example, to receive unstructured clinical notes or radiology reports that contain relevant information about the patient's condition. In such embodiments, the classifier module 113 uses natural language processing techniques like text mining, entity recognition, and sentiment analysis to extract relevant data from these sources. In addition, the testing system 101 and data acquisition module 111 may integrate with electronic health record (EHR) databases or laboratory information systems to retrieve pre-defined criteria for diagnostic tests based on age-specific guidelines and medical history. This allows the risk assessment module to consider individual patient characteristics when generating a comprehensive risk profile. In one embodiment, the testing system 101 conducts diagnostic tests on each patient based on pre-defined criteria stored in an electronic health record (EHR) database. Unstructured data generated by these tests is then received by a data acquisition module 111, which converts this raw information into structured binary or linear format using natural language processing techniques like text mining (e.g., optical character recognition and text-based PDF extraction), entity recognition, and sentiment analysis.

In another embodiment, the classifier module 113 may review each individual data source to convert unstructured data from various sources such as MRI reports, pathology results, operative notes, office records, etc. For example, in prostate cancer cases, values of 0 or 1 are assigned based on whether extracapsular extension is present (binary) or counts the number of biopsy cores with grade-group 4 or 5 disease. In one embodiment, the classifier module 113 may employ machine learning algorithms that may be trained using large datasets and adjusted for individual patients' characteristics through active learning. In some embodiments, these models may incorporate physician-provided criteria to determine outputted structured data.

The provider interface system 105 prioritizes data collection from various sources using algorithms that assess reliability of each input source and may weigh certain data more heavily based on clinical information such as demographics, medical history, laboratory results, imaging studies, pathology reports, operative notes, etc. to determine a risk profile. In some embodiments, provider interface system 105 may use simple binary “yes” or “no” outputs for displaying risk profiles. In other embodiments, the provider interface system 105 may also use visualization techniques such as heat maps, scatter plots and 3D models to present comprehensive risk profiles in an intuitive format for easy interpretation by healthcare professionals. In some embodiments, patient-specific factors like age, medical history, lifestyle habits, family history genetic predisposition environmental exposures etc. may influence analysis through incorporation of these variables into machine learning models. In genitourinary-oncology the use of risk-groups are used to identify patients who would benefit from certain treatments. For example, in prostate cancer high-risk prostate cancer is a term commonly used in scientific papers and national guidelines to describe a man prostate cancer based on predefined variables such as biopsy results, Prostate specific antigen levels, and the presence of extracapsular extension. Multiple independent unstructured data points are reviewed to determine ones risk-group. Currently, the National Comprehensive Cancer Networks (NCCN) definition of high-risk prostate cancer is having at least one of the following: prostate specific antigen level greater than 20 ng/mL, extracapsular extension, or a Gleason Group of 4 or 5 on prostate biopsy. Data received from the testing system is unstructured. The risk analysis module 103 converts this data into a structured format allowing the classifier module 113 to analysis the data to find patients who meet criteria for high-risk disease.

In addition, the provider interface system 105 may incorporate quality control measures, such as data validation checks peer review processes and continuous monitoring performance metrics against established benchmarks to account for potential biases in test results or other factors that may impact cancer risk groups and treatments, such as further diagnostic tests and therapeutics.

The classifier module 113 is a useful component of the patient management system that may employ machine learning algorithms to analyze structured data and produce accurate risk profiles for cancer patients. In one embodiment, the classifier module 113 uses supervised learning techniques where large datasets are trained on clinical information such as demographics, medical history, laboratory results, imaging studies, pathology reports, operative notes, and other relevant factors. The algorithm is configured to identify patterns in this data that correlate with increased or decreased risks of certain cancer risk-groups. Additionally, embodiments may include an analysis module using text mining, entity recognition, and sentiment analysis to analyze unstructured clinical notes or radiology reports and provide structured data. In yet another embodiment, the classifier module 113 may be configured with physician-provided criteria that determine outputted structured data. This allows for customization of risk assessment models based on individual patient characteristics. Furthermore, embodiments may include a knowledge base regularly updated with new evidence-based recommendations from reputable sources such as professional societies or government agencies to ensure accuracy and reliability in patient profiling. In addition, machine learning algorithms may be used in combination with natural language processing techniques like text mining and sentiment analysis to extract relevant information from unstructured clinical notes or radiology reports without requiring manual input. This enables healthcare professionals to focus on high-value tasks while ensuring accurate risk assessment for patients.

In certain embodiments, the classifier module 113 may be integrated into electronic health records (EHRs) systems allowing for seamless data exchange between different healthcare organizations. In other instances, it may use cloud-based platforms that enable real-time data synchronization across multiple providers. The algorithm is configured to handle missing values or incomplete patient records by using imputation methods such as statistical models of the underlying distribution.

The provider interface system 105 is a useful component of the patient management system 100 according to the present disclosure that enables seamless data collection and analysis from various sources to generate comprehensive risk profiles for cancer patients. In some embodiments, the provider interface system 105 may use simple binary “yes” or “no” outputs based on a threshold score for displaying risk profiles such as indicating high-risk patient populations. In other instances, it may employ visualization techniques like heat maps and scatter plots to present comprehensive risk profiles in an intuitive format for easy interpretation. In one embodiment, provider interface system 105 utilizes algorithms to prioritize data collection based on the reliability of each input source, such as electronic health records systems, laboratory information systems, radiology imaging platforms, or other relevant clinical databases.

In certain embodiments, the provider interface system 105 may be integrated into clinical decision support systems (CDSSs) that provide real-time feedback on treatment options based on individualized risk assessment results. This enables healthcare professionals to make informed decisions about patient care while ensuring accurate identification of risk groups, further diagnostic tests and therapeutics and effective management of cancer patients. In yet another embodiment, provider interface system 105 employs machine learning models trained on large datasets of structured clinical information to identify patterns and predict future risk based on individual patients' characteristics. These models may incorporate active learning techniques involving human-in-the-loop validation and feedback mechanisms to ensure accuracy and reliability in patient profiling.

Furthermore, provider interface system 105 may utilize collaborative tools like shared electronic health records or cloud-based platforms that enable real-time data exchange and synchronization across healthcare organizations. This allows multiple providers to contribute to a single patient's structured dataset while maintaining confidentiality and security standards.

A risk profile 205 is a comprehensive and individualized assessment of a patient's cancer based on their unique characteristics, medical history, laboratory results, imaging studies, pathology reports, operative notes, and other relevant factors. In one embodiment, the system generates this valuable information by converting unstructured data from various sources into structured binary or linear format using a classifier module 113. For instance, in prostate cancer cases, classifier module 113 assigns values of 0 or 1 based on whether extracapsular extension is present, counts the number of biopsy cores with grade-group 4 or 5 disease, and reviews pathology reports for grade-group 4 or 5 disease. In another embodiment, the classifier module 113 incorporates machine learning algorithms that are trained on large datasets to identify patterns in patient data and predict future risk based on historical trends.

In yet another embodiment, the provider interface system 105 prioritizes data collection from various sources using algorithms that assess the reliability of each input source, such as EHR systems, laboratory information systems, and radiology imaging platforms. The provider interface system 105 may also weigh certain data more heavily in consideration of clinical information, including demographics, medical history, laboratory results, imaging studies, pathology reports, operative notes, and other relevant factors. In still other embodiments, the provider interface system 105 may include quality control measures such as data validation checks, peer review processes, and continuous monitoring of performance metrics against established benchmarks. Additionally, machine learning algorithms may be integrated into provider interface system 105 analysis for continuous improvement and refinement of risk assessment models based on new data inputs through active learning techniques involving human-in-the-loop validation and feedback mechanisms to ensure accuracy and reliability in patient profiling.

The care planning process utilizes individualized risk profiles generated by the provider interface system 105 to provide the risk profile 205 and the resulting personalized treatment recommendations for cancer patients 109. Moreover, the system may incorporate a knowledge base that is regularly updated with new evidence-based recommendations from reputable sources like professional societies or government agencies. This ensures that provider interface system 105 remains current and effective in generating accurate risk profiles for identification of cancer risk groups and further diagnostic tests and therapeutics, including personalized treatment planning.

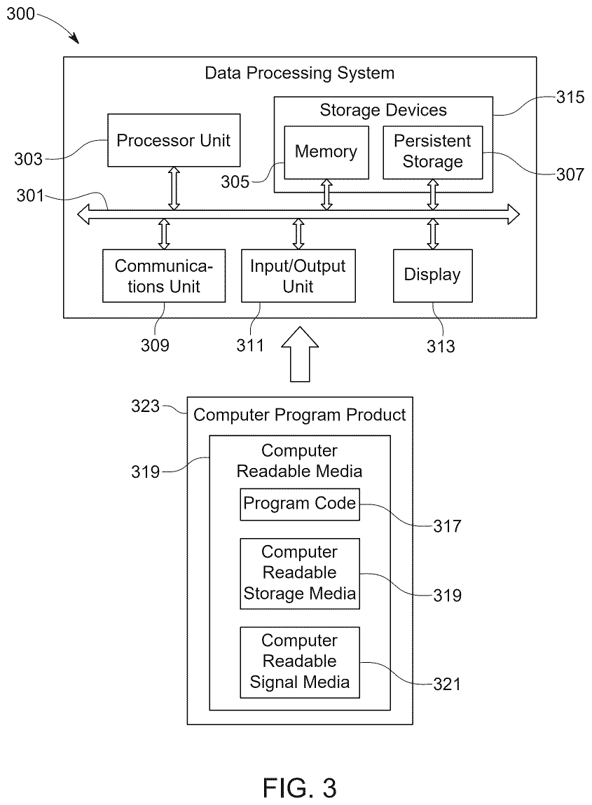

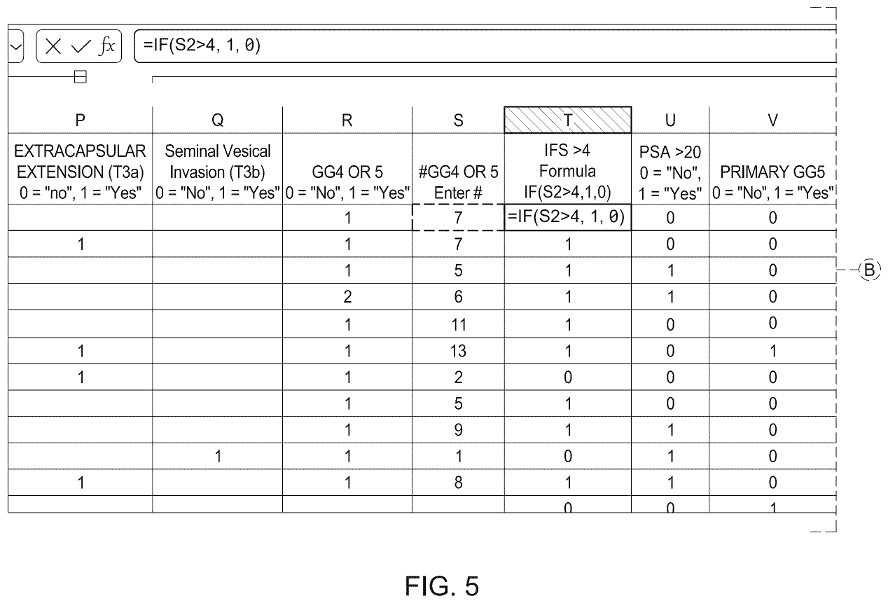

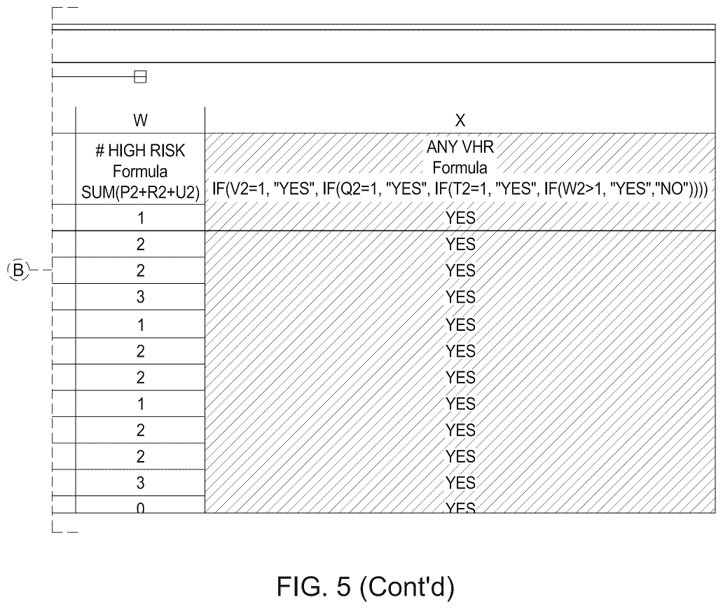

FIG. 3 shows an exemplary illustration of a data processing system 300 suitable for use as components of the patient management system 100, including, but not limited to, the risk analysis module 103, the provider interface 105, the data acquisition module 111, the classifier module 113, and/or the analysis module 115. In this illustrative example, data processing system 300 may include communications fabric 301, which provides communications between processor unit 303, memory 305, persistent storage 307, communications unit 309, input/output (I/0) unit 311 and display 313. While FIG. 5 shows various elements including processor unit 303, memory 305, persistent storage 307, communications unit 309, input/output (I/0) unit 311, and display 313, some or all of the elements may be present for particular configurations of the patient management system 100. The utilization or particular components is dependent upon the functionality needed for a particular risk analysis module 103, provider interface 105, data acquisition module 111, classifier module 113, and/or analysis module 115.

Processor unit 303 may be one or a number of processors, a multi-processor core, or some other type of processor, depending on the particular implementation. A number, as used herein with reference to an item, means one or more items. Further, processor unit 303 may be implemented using a number of heterogeneous processor systems in which a main processor is present with secondary processors on a single chip. As another illustrative example, processor unit 303 may be a symmetric multi-processor system containing multiple processors of the same type.

Memory 305 and persistent storage 307 are examples of storage devices 315. A storage device 315 is any piece of hardware that is capable of storing information, such as, for example, without limitation, data, program code in functional form, and/or other suitable information either on a temporary basis and/or a permanent basis. Storage devices 315 may also be referred to as computer readable storage devices in these examples. Memory 305, in these examples, may be, for example, a random access memory or any other suitable volatile or non-volatile storage device. Persistent storage 307 may take various forms, depending on the particular implementation.

For example, persistent storage 307 may contain one or more components or devices 105. For example, persistent storage 307 may be a hard drive, a flash memory, a rewritable optical disk, a rewritable magnetic tape, or some combination of the above. The media used by persistent storage 307 also may be removable. For example, a removable hard drive may be used for persistent storage 307.

Communications unit 309, in these examples, provides for communications with other data processing systems or devices. In these examples, communications unit 309 is a network interface card. Communications unit 309 may provide communications through the use of either or both physical and wireless communications links.

Input/output (I/0) unit 311 allows for input and output of data with other devices 105 that may be connected to data processing system 300. For example, input/output (I/0) unit 311 may provide a connection for user input through a keyboard, a mouse, and/or some other suitable input device. Further, input/output (I/0) unit 311 may send output to a printer. Display 313 provides a mechanism to display information to a user.

Instructions for the operating system, applications, and/or programs may be located in storage devices 315, which are in communication with processor unit 303 through communications fabric 301. In these illustrative examples, the instructions are in a functional form on persistent storage 307. These instructions may be loaded into memory 305 for execution by processor unit 303. The processes of the different embodiments may be performed by processor unit 303 using computer implemented instructions, which may be located in a memory, such as memory 305.

These instructions are referred to as program code 317, computer usable program code, or computer readable program code that may be read and executed by a processor in processor unit 303. The program code 317 in the different embodiments may be embodied on different physical or computer readable storage media 319, such as memory 305 or persistent storage 307.

Program code 317 is located in a functional form on computer readable storage media 319 that is selectively removable and may be loaded onto or transferred to data processing system 300 for execution by processor unit 303. Program code 317 and computer readable storage media 319 form computer program product 323 in these examples. In one example, computer readable storage media 319 may be computer readable storage media 319 or computer readable signal media 321. Computer readable storage media 319 may include, for example, an optical or magnetic disk that is inserted or placed into a drive or other device 105 that is part of persistent storage 307 for transfer onto a storage device, such as a hard drive, that is part of persistent storage 307. Computer readable storage media 319 also may take the form of a persistent storage, such as a hard drive, a thumb drive, or a flash memory, that is connected to data processing system 300. In some instances, computer readable storage media 319 may not be removable from data processing system 300.

Alternatively, program code 317 may be transferred to data processing system 300 using computer readable signal media 321. Computer readable signal media 321 may be, for example, a propagated data signal containing program code 317. For example, computer readable signal media 321 may be an electromagnetic signal, an optical signal, and/or any other suitable type of signal. These signals may be transmitted over communications links, such as wireless communications links, optical fiber cable, coaxial cable, a wire, and/or any other suitable type of communications link. In other words, the communications link and/or the connection may be physical or wireless in the illustrative examples.

In some illustrative embodiments, program code 317 may be downloaded over a network to persistent storage 307 from another device 105 or data processing system 300 through computer readable signal media 321 for use within data processing system 300. For instance, program code stored in a computer readable storage medium in a server data processing system may be downloaded over a network from the server to data processing system 300. The data processing system 300 providing program code 317 may be a server computer, a client computer, or some other device capable of storing and transmitting program code 317.

The different components illustrated for data processing system 300 are not meant to provide architectural limitations to the manner in which different embodiments may be implemented. The different illustrative embodiments may be implemented in a data processing system 300 including components in addition to or in place of those illustrated for data processing system 300. Other components shown in FIG. 5 can be varied from the illustrative examples shown. The different embodiments may be implemented using any hardware device or system capable of running program code 317. As one example, the data processing system 300 may include organic components integrated with inorganic components and/or may be comprised entirely of organic components excluding a human being. For example, a storage device may be comprised of an organic semiconductor.

In another illustrative example, processor unit 303 may take the form of a hardware unit that has circuits that are manufactured or configured for a particular use. This type of hardware may perform operations without needing program code 317 to be loaded into a memory from a storage device to be configured to perform the operations.

For example, when processor unit 303 takes the form of a hardware unit, processor unit 303 may be a circuit system, an application specific integrated circuit (ASIC), a programmable logic device, or some other suitable type of hardware configured to perform a number of operations. With a programmable logic device, the device 105 is configured to perform the number of operations. The device 105 may be reconfigured at a later time or may be permanently configured to perform the number of operations. Examples of programmable logic devices include, for example, a programmable logic array, programmable array logic, a field programmable logic array, a field programmable gate array, and other suitable hardware devices. With this type of implementation, program code 317 may be omitted because the processes for the different embodiments are implemented in a hardware unit.

In still another illustrative example, processor unit 303 may be implemented using a combination of processors found in computers and hardware units. Processor unit 303 may have a number of hardware units and a number of processors that are configured to run program code 317. With this depicted example, some of the processes may be implemented in the number of hardware units, while other processes may be implemented in the number of processors.

The different illustrative embodiments can take the form of an entirely hardware embodiment, an entirely software embodiment, or an embodiment containing both hardware and software elements. Some embodiments are implemented in software, which includes but is not limited to forms such as, for example, firmware, resident software, and microcode.

Furthermore, the different embodiments can take the form of a computer program product accessible from a computer usable or computer readable medium providing program code 317 for use by or in connection with a computer or any device 105 or system that executes instructions. For the purposes of this disclosure, a computer usable or computer readable medium can generally be any tangible apparatus that can contain, store, communicate, propagate, or transport the program for use by or in connection with the instruction execution system, apparatus, or device.

The computer usable or computer readable medium can be, for example, without limitation an electronic, magnetic, optical, electromagnetic, infrared, or semiconductor system, or a propagation medium. Non-limiting examples of a computer readable medium include a semiconductor or solid-state memory, magnetic tape, a removable computer diskette, a random access memory (RAM), a read-only memory (ROM), a rigid magnetic disk, and an optical disk. Optical disks may include compact disk-read only memory (CD-ROM), compact disk-read/write (CD-R/W), and DVD.

Further, a computer usable or computer readable medium may contain or store a computer readable or computer usable program code 317 such that when the computer readable or computer usable program code is executed on a computer, the execution of this computer readable or computer usable program code causes the computer to transmit another computer readable or computer usable program code over a communications link. This communications link may use a medium that is, for example, without limitation, physical or wireless.

A data processing system 300 suitable for storing and/or executing computer readable or computer usable program code 317 will include one or more processors coupled directly or indirectly to memory elements through a communications fabric, such as a system bus. The memory elements may include local memory employed during actual execution of the program code, bulk storage, and cache memories which provide temporary storage of at least some computer readable or computer usable program code 317 to reduce the number of times code may be retrieved from bulk storage during execution of the code.

Input/output or I/O devices 135 can be coupled to the system either directly or through intervening I/O controllers. These devices 105 may include, for example, without limitation, keyboards, touch screen displays, and pointing devices. Different communications adapters may also be coupled to the system to enable the data processing system 300 to become coupled to other data processing systems 300 or remote printers or storage devices through intervening private or public networks. Non-limiting examples of modems and network adapters are just a few of the currently available types of communications adapters.

The system according to the present disclosure may easily be integrated into a software program (Example—web based subscription service). This allows for widespread use of the system according to the present disclosure. In addition, machine learning and/or artificial intelligence may be utilized to automate the process further. For example, magnetic resonance imaging reports may be uploaded and machine learning/artificial intelligence may “read” the file looking for keywords to determine if extracapsular extension pT3a or seminal vesical pT3b invasion is present. Likewise, machine-learning or artificial intelligence may be used to “read” through prostate biopsy reports.

Numerous downstream applications are possible from the aggregated data from the system according to the present disclosure. Two examples include utilizing the data to help clinical trial enrollment and clinical practices identify patients more readily. Concerning clinical trials, the longer period takes a pharmaceutical company or organization to complete patient enrollment “meet accrual” the higher expenses will be. The system according to the present disclosure allows for participating clinical trials sites to identify potential clinical trial patients faster and more efficiently. This results in lower expenses to the clinical trial company/organizer and perhaps lower drug prices for patients. Similarly, by being able to more efficiently identify patients in a clinical setting the system according to the present disclosure improves patient outcomes and a practices revenue stream.

For example, in the instance of prostate cancer, multiparametric imaging (MRI's) may be reviewed for extracapsular extension. If no extracapsular extension is reported a value of “0” is assigned. If extracapsular extension is present a value of “1” is assigned. Likewise, this is performed for seminal vesicle invasion/involvement “0” not involved “1” involved/invasion, if pathology shows grade-group 4 or 5 disease “0” if not present and “1” if present, PSA value greater than 20 “0” if the PSA is less than 20 and “1” if 20 or greater, and if the primary Gleason score is 5, “0” if not 5 and “1” if 5. For linear data the data source is reviewed, and a “count” is entered. For example, if a prostate biopsy has 12 samples and 6 of those samples have a grade-group of 4 or 5 disease the count would be “6”. This linear number is entered into a dedicated field and transformed into binary data by asking is the total number of biopsy cores with grade-group 4 or 5 disease>4 “0” no “1” yes. By converting all the unstructured data into a binary format, the created structure that can be used for analysis.

In the instance of bladder cancer, pathology reports are reviewed for grade (low grade or high grade), presence of carcinoma in-situ. Operative reports are reviewed for the “Current Procedural Terminology” code (CPT) to determine bladder tumor size and number of tumors as well as date of last reoccurrence of bladder cancer. This data is transformed from unstructured data into semi-structured data in the form of binary or continuous data.

Pathology reports are reviewed for bladder tumor grade, low-grade is assigned a value of “0” and high-grade or CIS “1”, The presence of carcinoma in-situ (CIS) “0” not present, “1” present, depth of tumor invasion “0” Ta non-invasive, “1” T1 invasive into submucosa or CIS, “2” T2 invasive into muscularis propria, “MIBC” muscle invasive bladder cancer, and very high-risk features “0” no very high risk features, “3” yes very high risk features present such as variant histology, Lympovascular invasion, or Prostate urethral Invasion. Operative reports are reviewed for Current Procedural Terminology codes which identify size of the bladder tumor. CPT codes 52224 and 52234 are assigned a value of “0” and CPT codes 52235 and 52240 are assigned a value of “1”. Operative and office notes are also reviewed for the number of lesions during the procedure “0” if solitary, “1” if multifocal. Date of last bladder cancer reoccurrence/treatment “0” not within a year, “1” within a year. Office notes are also reviewed for previous Bacillus Calmette-Guerin (BCG) use. Specifically, a count of how many doses administered. BCG is administered in a regimented way on an “induction schedule”, six treatments one a week for six weeks, or “maintenance” treatments, 3 treatments one a week for three weeks at set intervals. A count (1, 2, 3, 4, 5, 6 . . . ) is performed for how many of the induction doses (1-6) were given and maintenance doses (1-3) were given. For example, if a patient received 5 of 6 induction doses the count would be 5.

### EXAMPLES

### Prostate Cancer Patient Management System

Clinical information is obtained following a prostate biopsy (CPT Code Billing). Clinical information includes, for example, clinical Information, age, and PSA. The Prostate Biopsy Pathology Report includes unstructured data including highest Gleason Group/Score, number of biopsy cores greater than or equal to GG4 disease and any primary GS 5 disease. The Magnetic Resonance Imaging (MRI) report includes unstructured data including strong language (Frank, obvious, gross) concerning for extracapsular extension (T3a) disease and strong language (frank, obvious, gross) concerning for seminal vesical invasion (T3b) disease.

The classifier module utilized in this example is a spreadsheet file wherein data is provided to predefined cells which converts unstructured data into structured data. Thereafter, the provider interface system displays, as a result of an aggregation of the structured data, patients having either intermediate risk, high-risk, and very high-risk prostate cancer.

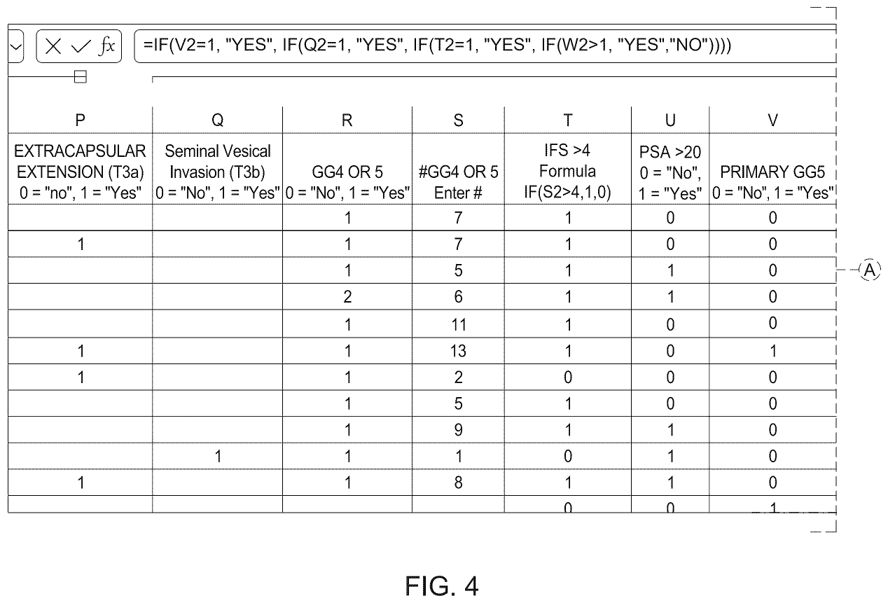

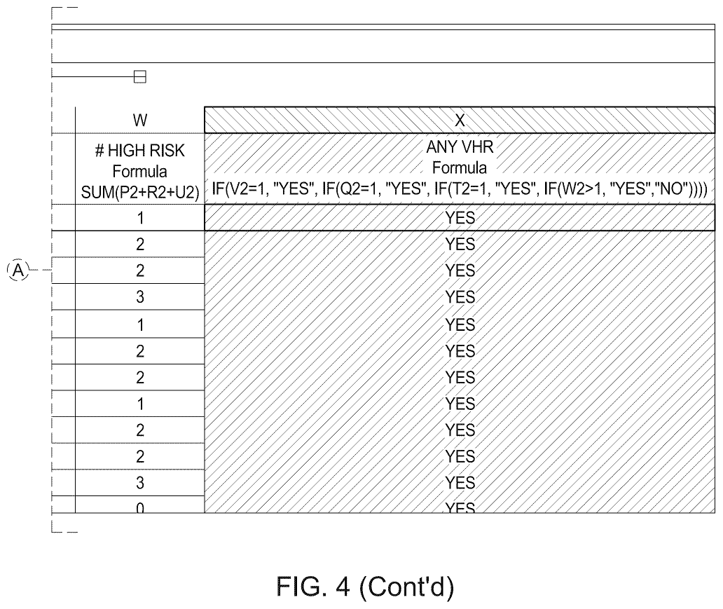

FIGS. 4-7 illustrate the conversion for the unstructured data to the structured data and the output of the risk profile (e.g., “VHR” (very high risk)) for prostate cancer.

### Prostate Cancer Evaluation

Below FIG. 4 shows an overview of system according to an embodiment of the present disclosure. The inputs into the cells are the semi-structed data. For example, column “P” is looking at extracapsular extension pT3a disease. An assigned value of “0 or blank” means the magnetic resonance imaging showed no obvious extracapsular extension and a value of “1” means obvious extracapsular extension was seen. In column “Q” the same magnetic resonance imaging is reviewed for seminal vesicle invasion pT3b disease. An assigned value of “0 or blank” means the magnetic resonance imaging showed no obvious seminal vesicle invasion and a value of “1” means obvious seminal vesicle invasion was seen. Column “R” transforms the prostate biopsy pathology into binary data. If the prostate biopsy shows no grade-group 4 or 5 disease a value of “0” is assigned. If the prostate biopsy shows any grade-group 4 or 5 disease a value of “1” is assigned. Column “S” transforms the number of prostate biopsy cores with grade-group 4 or 5 disease into a linear number (1, 2, 3, 4 . . . ) this is a count of biopsy cores, for example if 7 of 12 cores had grade-group 4 or 5 disease the count value would be 7. In column “T” the data from column “S” is transformed into binary data (please see FIG. 5). If the number in column “S” is less than or equal to 4 “0” is assigned. If the number in column “S” is 5 or greater “1” is assigned. In column “U” Prostate specific antigen (PSA) levels are analyzed. If the PSA value is less than 20 “0” is assigned. If the PSA value is 20 or greater “1” is assigned. In column “V” the prostate biopsy pathology is reviewed looking at the Gleason Score. A Gleason score is made up of two numbers the primary and secondary number (3+3, 3+4, 4+3, 4+4, 4+5, 5+4, 5+5). If the primary Gleason score is less than 5 “0 or blank” is assigned. If the primary Gleason Score is 5 “1” is assigned.

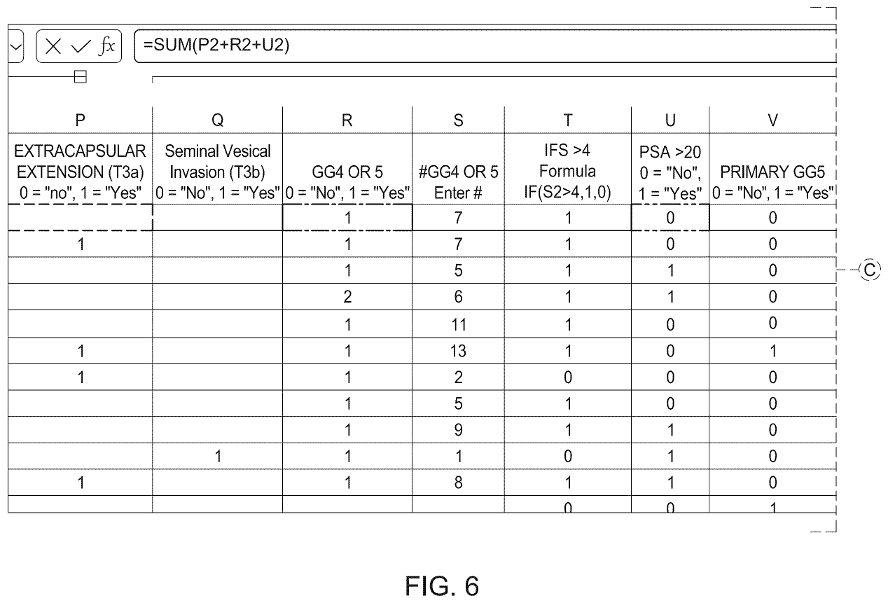

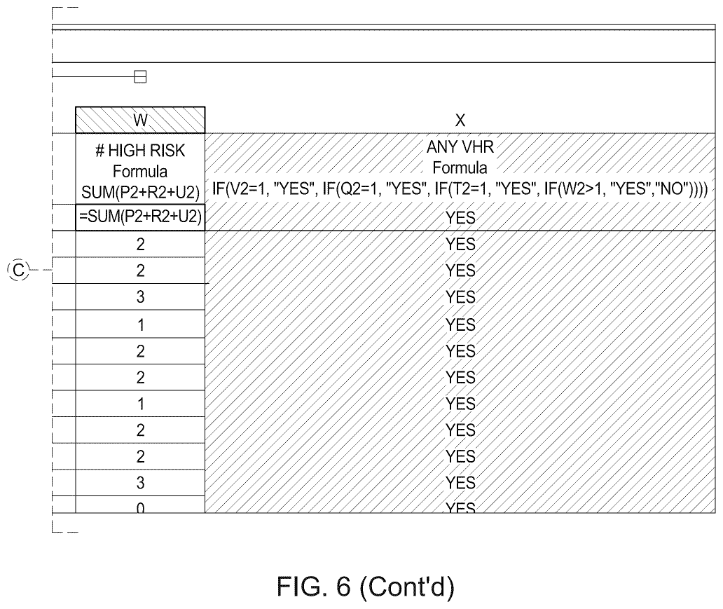

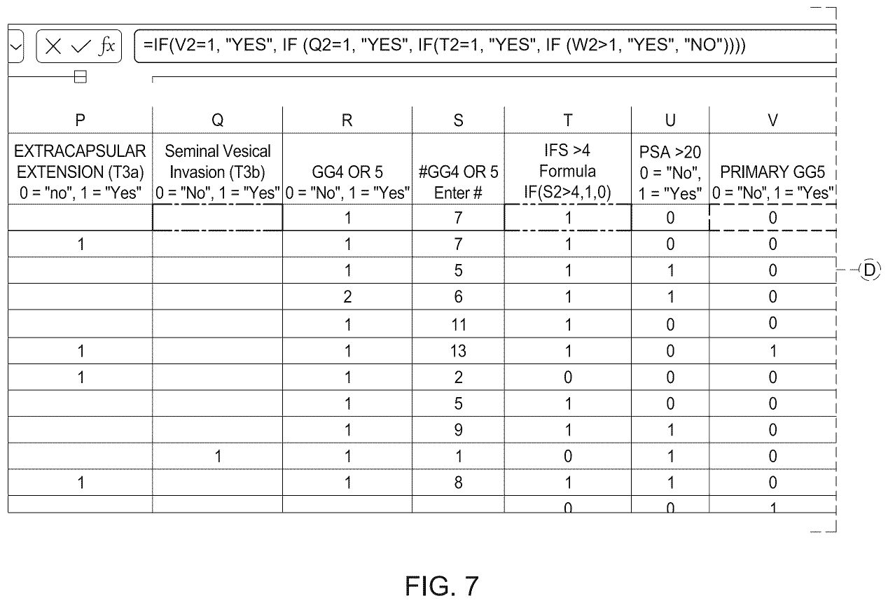

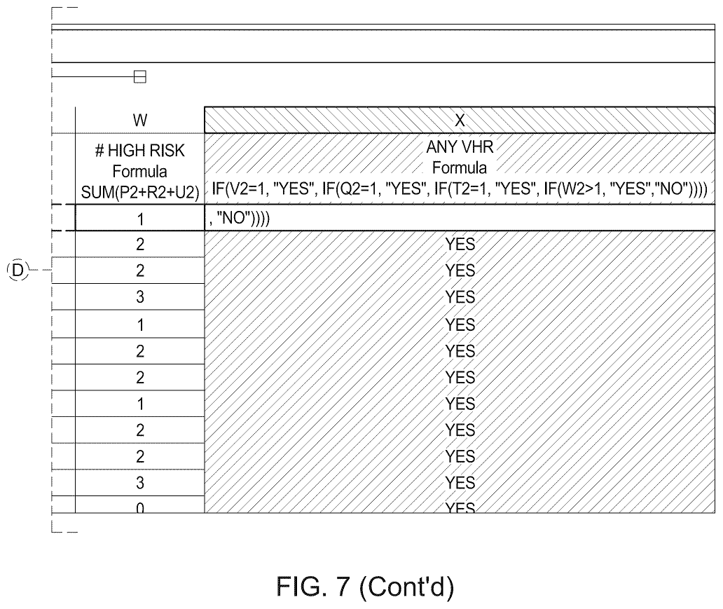

FIGS. 6 and 7 below demonstrate the analysis that is performed on the semi-structured data converted from multiple unstructured data sources. In FIG. 6 column “W” shows the system looking at high-risk prostate cancer features. If the value in column “W” (Sum of columns P, R, &U) is greater than or equal to one the patient has at least high-risk prostate cancer.

In FIG. 6 column “X” shows the system looking at very high-risk prostate cancer features. If the value in column “X” does not meet criteria for very high-risk prostate cancer a value of “NO” is assigned. If the value of in column “X” (which sums all the previously entered semi-structured data looking at very high-risk features) meets threshold a value of “YES” is assigned. Threshold for very high-risk prostate cancer is defined as having two or three high-risk features (column “W”) or a value of “1” in columns “Q, T, OR V”.

Patients that have either high or very-high risk prostate cancer qualify for treatment intensification with additional therapies and certain diagnostic tests. For example, in men with high or very high-risk prostate cancer germline testing (genetic testing) and more accurate imaging (Prostate specific membrane antigen CT/PET scan) can be utilized. Concerning treatment intensification several clinical trials are currently underway looking to see if adding novel therapies to men with high-risk prostate cancer improves outcomes such as metastasis free survival and overall survival. Men with very high-risk prostate cancer qualify for the same diagnostics as men with high-risk prostate cancer (germline and prostate specific membrane antigen CT/PET). However, in men with very high-risk prostate cancer electing for radiation the current standard of care is to add treatment intensification with a novel hormone agent (abiraterone) and several clinical trials currently exist for these patients.

Proper identification of patients with high or very high-risk prostate cancer is important as the intensified diagnostics and treatments available to them can improve outcomes. From a business perspective, being able to identify these patients may allow for increased utilization of diagnostic tests and treatments leading to improved revenue.

### Bladder Cancer Patient Management System:

Clinical information is obtained following a bladder biopsy or Transurethral resection of bladder tumor. Clinical information includes demographics, such as age, previous bladder cancer diagnosis dates, previous Bladder Cancer treatments (BCG) (e.g., doses of BCG Induction and doses of BCG Maintenance. The Bladder Pathology report includes unstructured data including grade (low/high grade), presence of carcinoma in-situ, depth of invasion (e.g., Ta—mucosal only involvement, T1—Submucosal involvement, T2—Muscularis propria involvement), very High-Risk Features (e.g., variant histology, lymphovascular invasion, prostate urethral invasion), office/clinical/operative notes, current Procedural Terminology Code (CPT) to determine tumor size, number of lesions/tumors solitary vs multi-focal, time since last treatment and reoccurrence of bladder cancer, and previous BCG use.

The classifier module utilized in this example is a spreadsheet file wherein data is provided to predefined cells which converts unstructured data into structured data. Thereafter, the provider interface system displays, as a result of an aggregation of the structured data, the output of the risk profile (e.g., “VHR” (very high risk)) for bladder cancer.

Concerning bladder cancer, the following figures demonstrate how the unstructured data is transformed into semi-structured data and analyzed:

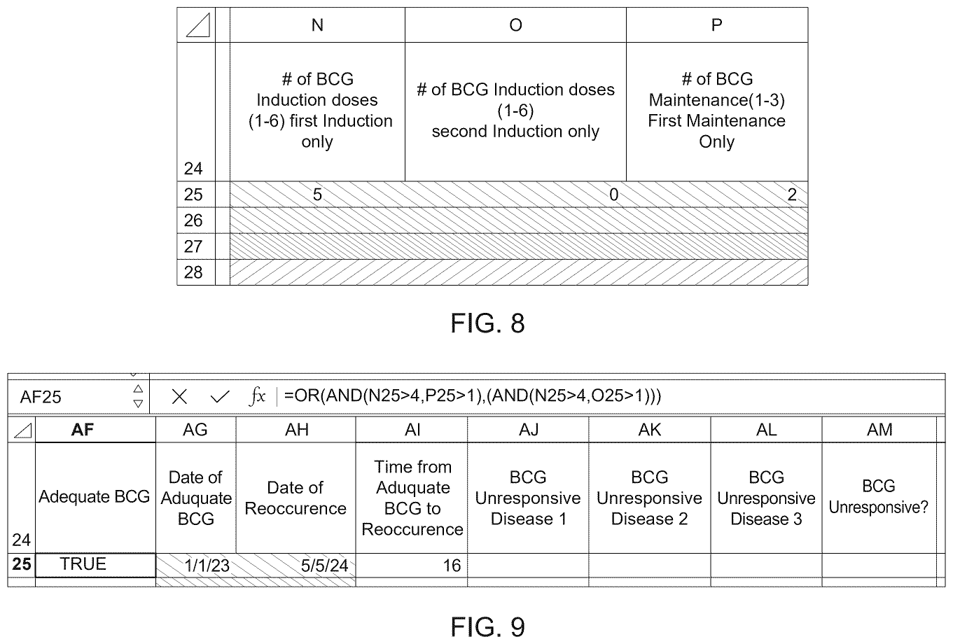

FIG. 8 demonstrates how we analyze previous BCG treatments and assess for “Adequate BCG”. For columns “N, O, P” a count of BCG doses is performed. This information is gathered from patient charts. In the example provided, this patient received five of six doses of a first induction course, zero doses of a second induction course, and two of three maintenance doses. Therefore, their counts are “N”=5, “O”=0, and “P”=2.

In FIG. 9, cell AF 26 is highlighted demonstrating the code assessing for “Adequate BCG”. The code pulls data from the cells demonstrated in FIG. 8. A result of “True” indicates adequate BCG while a result of “False” indicates adequate BCG was not given. In the example provided the patient did have adequate BCG. This is a very practical application. In patients that have not had adequate BCG, additional BCG should be considered. In patients that have had adequate BCG, additional BCG is unlikely to be beneficial and alternative therapies or enrollment in a clinical trial should be considered.

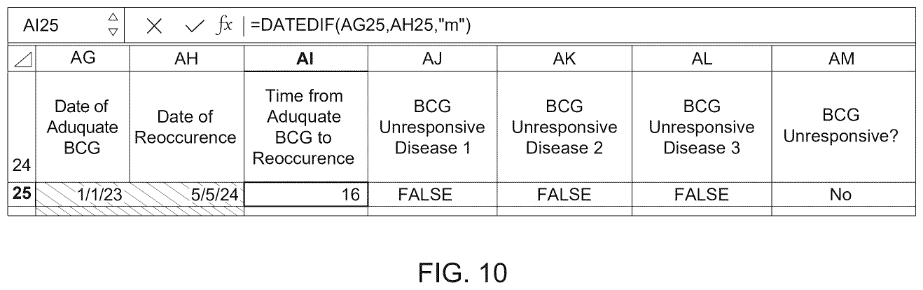

In FIG. 10, column “Time from adequate BCG to Reoccurrence” “AI” is highlighted with the corresponding code in the function (Fx) line. This column calculates the time in months between date of adequate BCG and date of reoccurrence. The function used is a commonly used function (=datedif(X, Y, “m”) but the result is used later to help determine a BCG unresponsive state which is a unique code and part of this program.

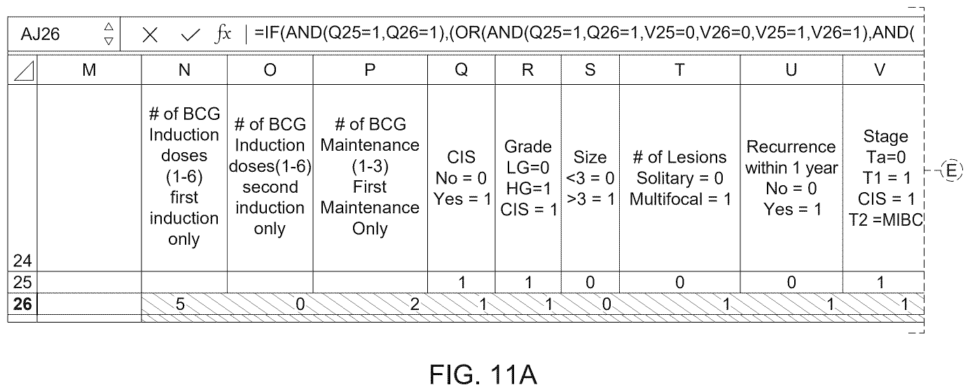

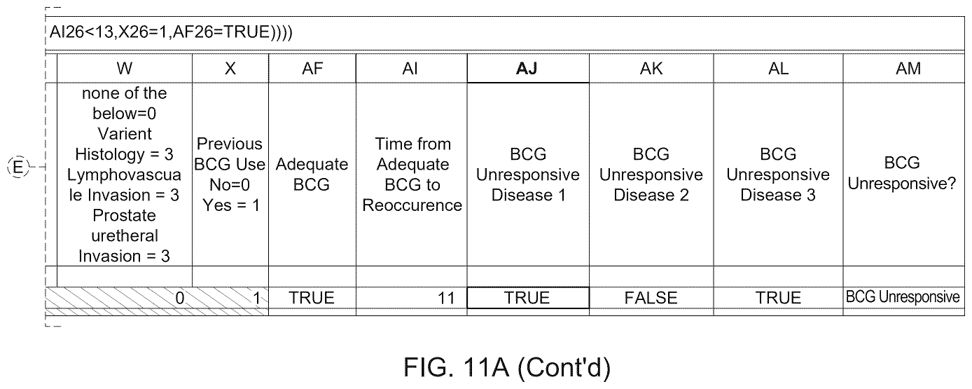

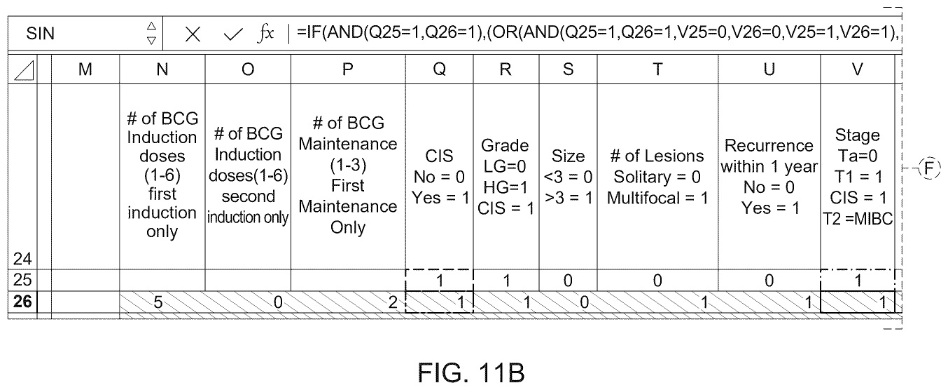

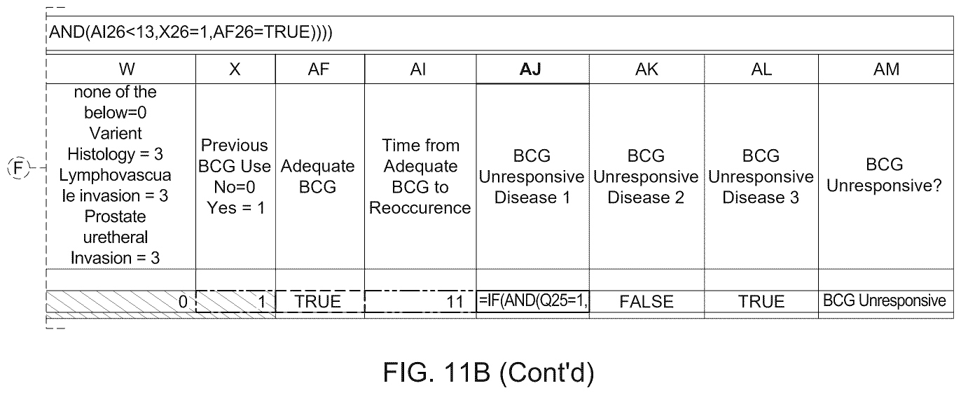

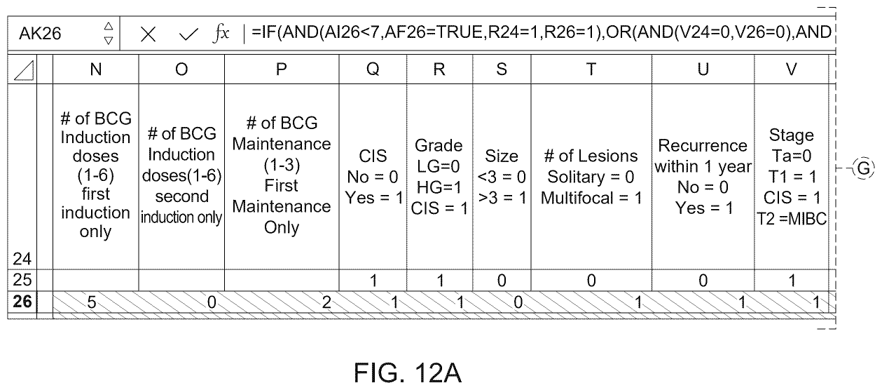

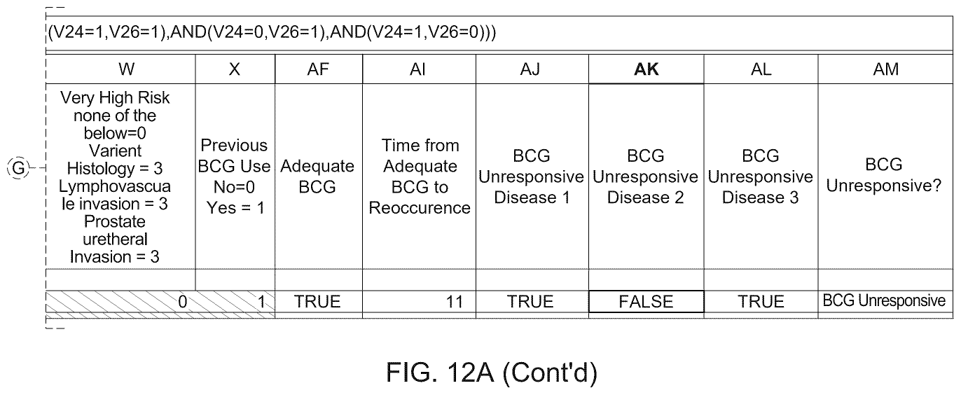

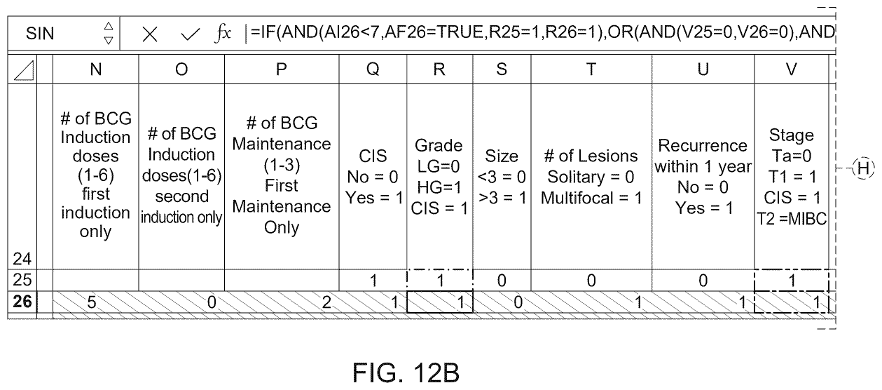

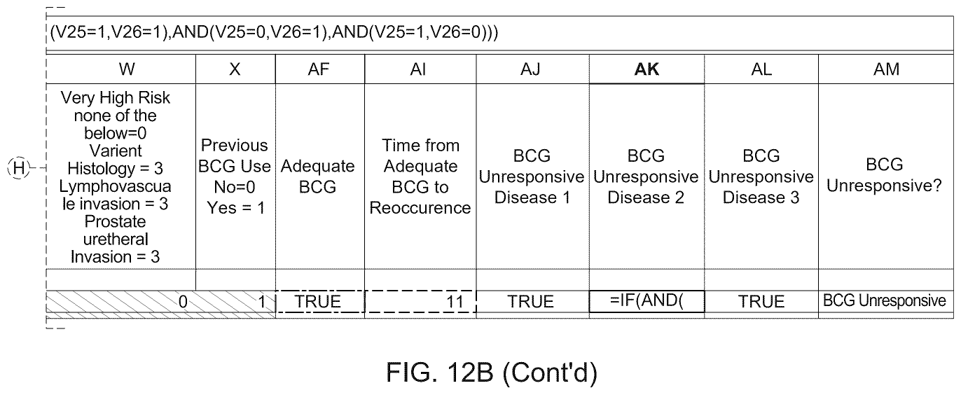

The Federal Food and Drug Administration (FDA) defines BCG unresponsive bladder cancer as one of the following: 1) Persistent or recurrent CIS alone OR persistent or recurrent CIS with recurrent Ta/T1 within 12 months of adequate BCG, 2) recurrent HG Ta/T1 within 6 months of adequate BCG, 3) T1 HG at first evaluation following induction BCG. FIGS. 11-13 demonstrate how we find patients with BCG unresponsive disease.

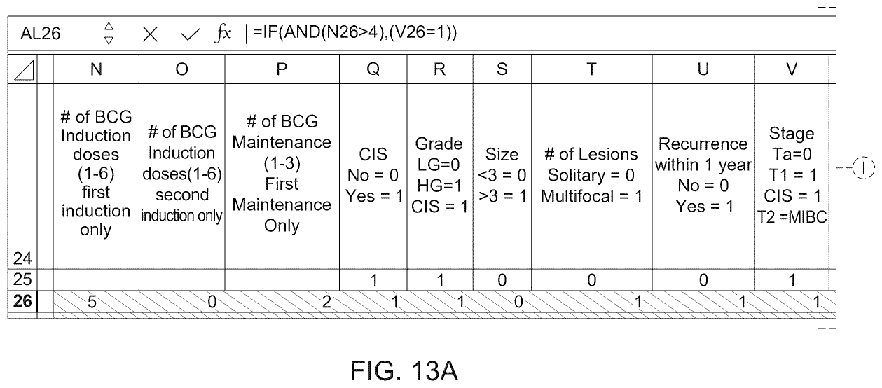

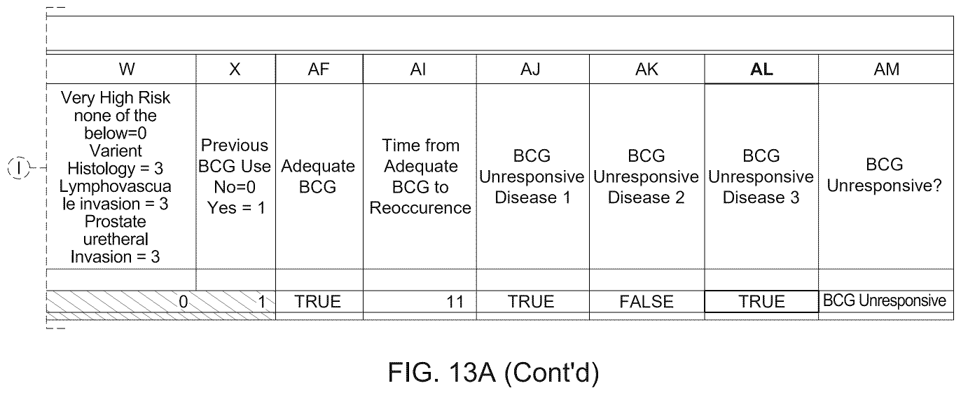

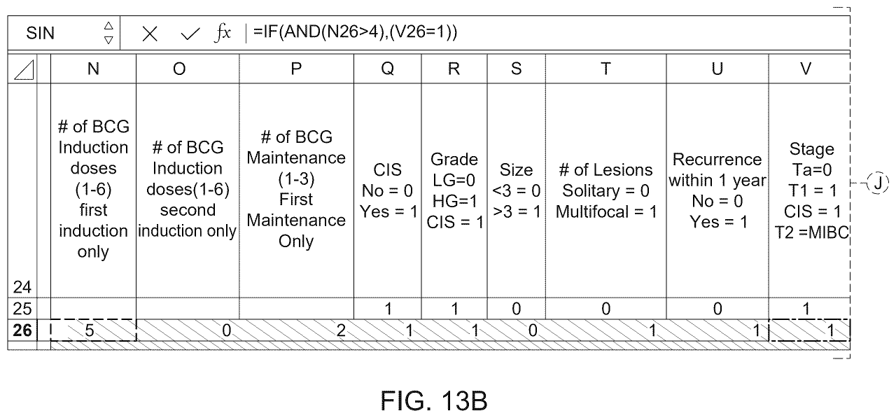

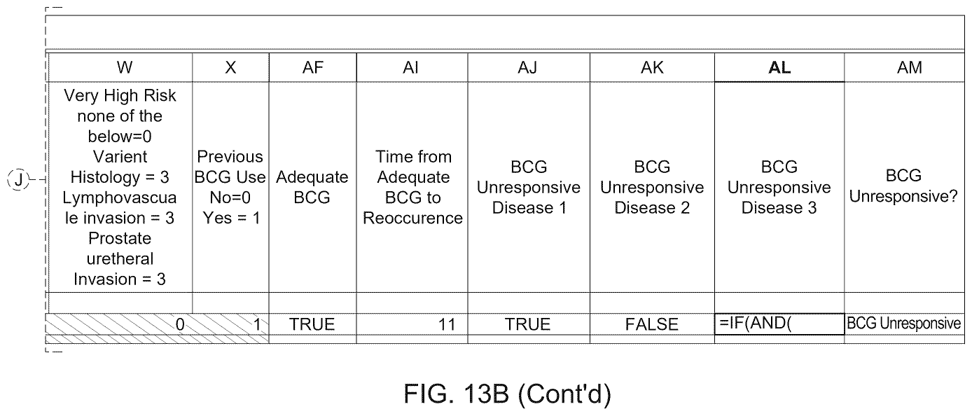

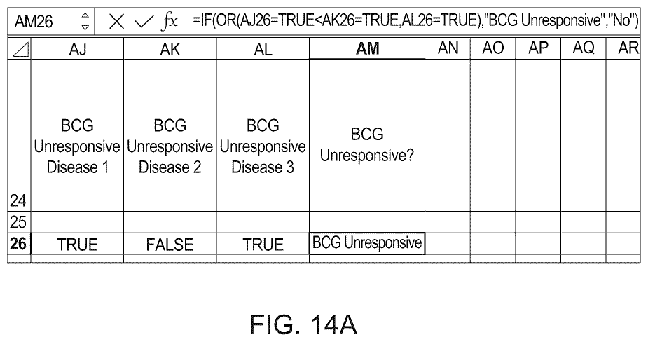

In FIG. 11a column “BCG Unresponsive 1” “AJ” is highlighted with the corresponding code in the function (Fx) line. This formula compares two rows of data (the same patient's data but at a different time period) to determine if they meet criteria of the FDA's definition of BCG unresponsive disease. FIG. 8b highlights the formula, column “Q” is used to determine if CIS was and is present at time of tissue sampling “0” no CIS present and “1” yes CIS present (Bladder biopsy or TURBT), column “V” is used to determine if Ta or T1 disease was and is present at time of tissue sampling “0” equals Ta disease “1” equals either T1 or CIS disease, column “X” is used to determine if BCG was previously used “0” no previous BCG and “1” yes previous BCG, column “AF” is used to see if adequate BCG was used (see FIG. 9), and column “AI” used to review the amount of time from adequate BCG to reoccurrence (see FIG. 10). In this scenario the criteria were meet therefore the product of the formula in column “AJ” is “TRUE”. Column BCG Unresponsive 2 “AK” looks to see if the second set of the FDA's criteria are meet. FIG. 11a is an overview of the function with the corresponding cells highlighted in FIG. 11b. Column “R” reports if high-grade disease was present both initially and at time of reoccurrence “0” Low-Grade and “1” High-Grade. Column “V” reports depth of invasion “0” Ta mucosal only disease, “1” T1 or submucosal disease or CIS, “MIBC” muscle invasive bladder cancer, column “AF” reports if adequate BCG was administered (FIG. 9), and column “AI” reports the time from adequate BCG to reoccurrence (FIG. 10). In this scenario criteria were not meet therefore the product of the formula in cell “AK” is “FALSE” is generated. To determine if the third criteria for BCG unresponsive disease is met column “AL” BCG Unresponsive 3 is used as demonstrated in FIG. 13a. The formula for column “AL” is shown in FIG. 13b. Column “AL” interprets data from columns “N” to see if BCG induction was administered and how many doses were given “<4”=no BCG induction and “>4”=yes BCG induction and column “V” to see if T1 disease is present “V=1”. In this scenario the criteria were met thus a value is column “AL” returns as “TRUE”. In order to determine if any of the three FDA's criteria were met column “AM” asks if any of the cells in the corresponding row in columns “AJ, AK, AL” are “TRUE”. As long as one cell is TRUE the output in column “AM” will be “BCG Unresponsive”. If all the criteria in cells “AJ, AK, AL” are false the output will be “NO” as demonstrated in FIGS. 14a and 14b.

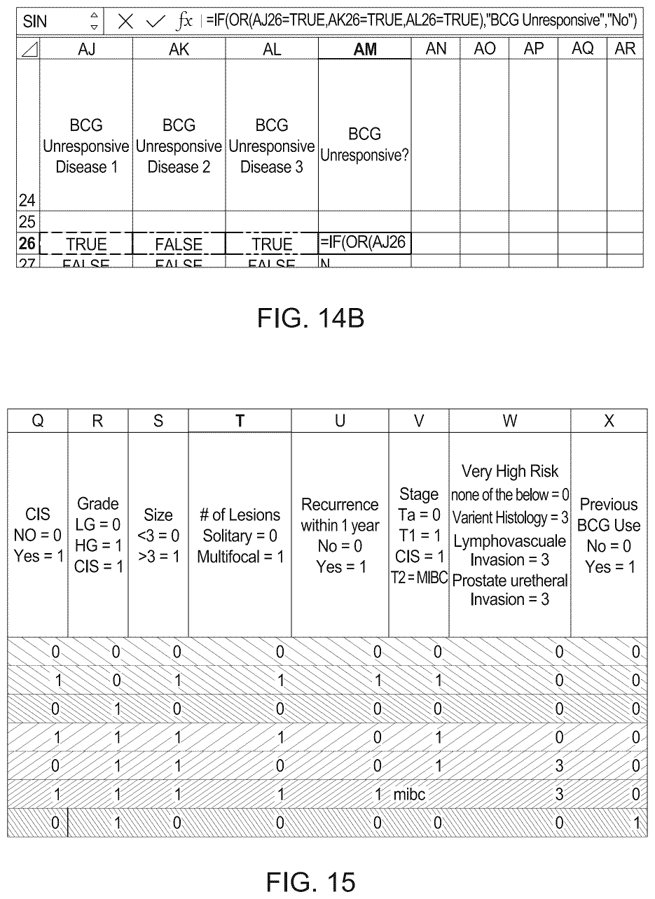

FIG. 15 demonstrates additional field inputs of semi-structured data. This data is obtained from pathology reports following a bladder biopsy or transurethral resection of a bladder tumor, office notes, and current procedural terminology codes (CPT). Several data points are derived from the pathology report included those in column “Q, R, V, and W”. Column “Q” asks if Carcinoma in-situ (CIS) is present on the pathology report a value of “0” indicates no CIS was present and “1” indicates CIS was present. Column “R” asks what tumor grade was reported on the pathology report. A value of “0” indicates low-grade while a value of “1” indicates the presence of high-grade or CIS. Column “V” transforms the reported depth of tumor invasion from the pathology report to numerical format. Concerning stage/depth of invasion “0” equals Ta or mucosal only/non-invasive disease, “1” equals CIS or invasion into the lamina propria, “MIBC” indicates muscle invasive bladder cancer or invasion into the muscularis propria. Column “W” input is obtained from the pathology report as well. Certain bladder cancer histology patterns are considered “variant” and known to be more aggressive, likewise, bladder cancer that demonstrates lymphovascular or prostatic urethral invasion have a worse prognosis and are therefore important to document. In column “W” the absence of any very high-risk features is recorded as “0”, if any very high-risk features (variant history, lymphovascular invasion, prostatic urethral invasion) are present a value of “3” is assigned.

Information inputted into column “S” is obtained from a combination of operative notes or common procedural terminology (CPT) codes used for billing. A CPT code of either “52224 or 52234” indicates a bladder tumor size of less than 3 centimeters and a value of “0” is assigned. A CPT code of “52240” indicates a bladder tumor size of greater than 5 centimeters and a value of “1” is assigned. A CPT code of “52235” indicates a bladder tumor size of 2-5 centimeters. Given that this CPT code, “52235”, overlaps the 3-centimeter threshold a review of the operative report is completed to assign the tumor size of less than or greater than 3 centimeters “0” and “1” respectively.

Information inputted into column “U” is obtained from office or operative notes. Bladder cancer is known to reoccur frequently. The timing or reoccurrence has important implications and thus important to capture.

During a repeat bladder biopsy or TURBT if a reoccurrence happens greater than 12 months from a previous reoccurrence/initial diagnosis a value of “0” is assigned. If a reoccurrence does occur within 12 months a value of “1” is assigned. Column “X” records is BCG has ever previously been used “0” no, “1” yes”.

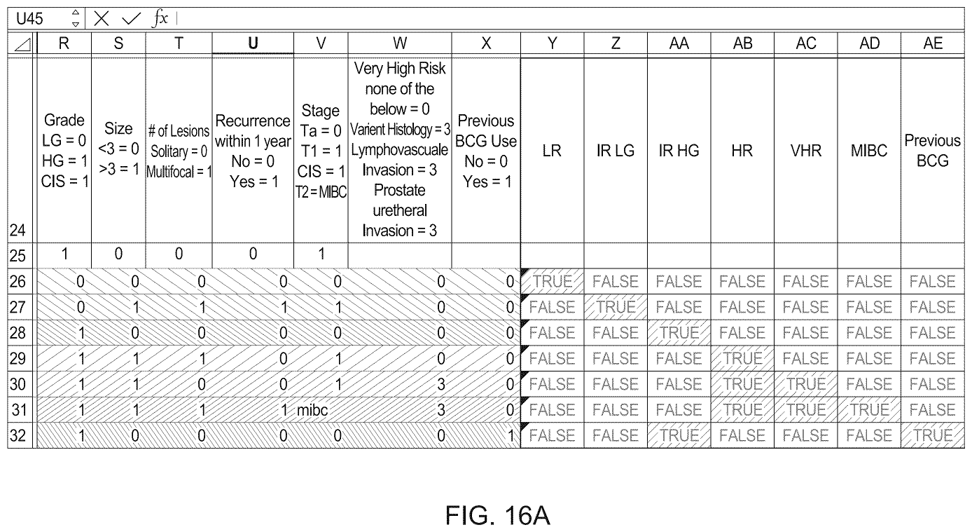

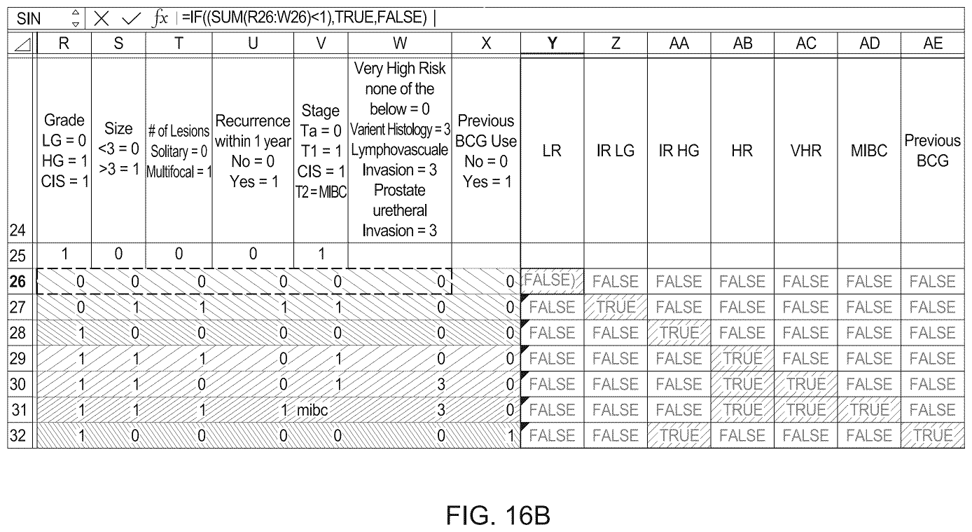

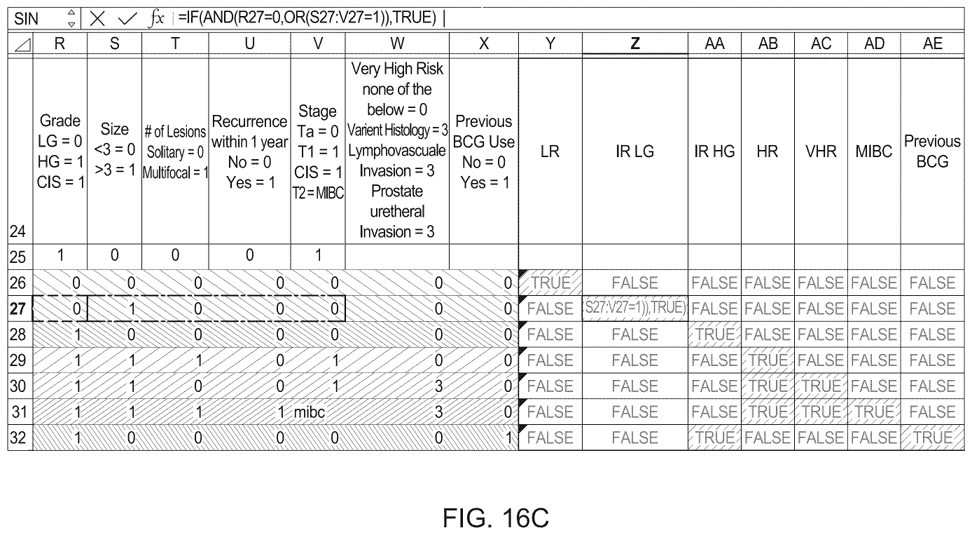

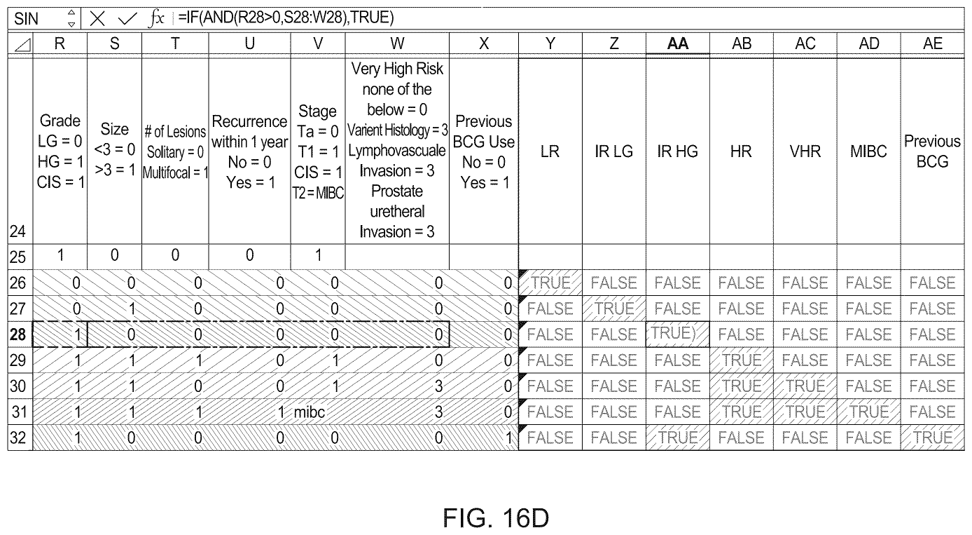

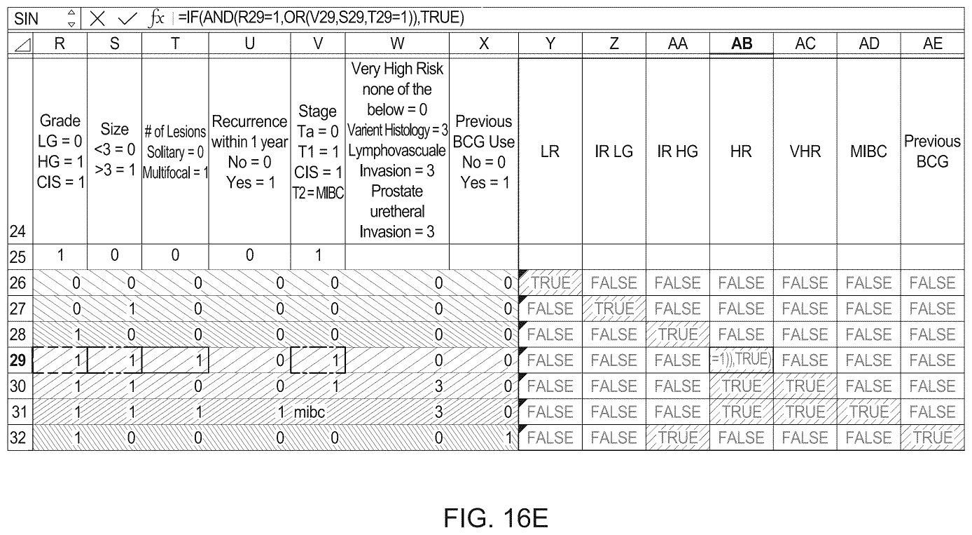

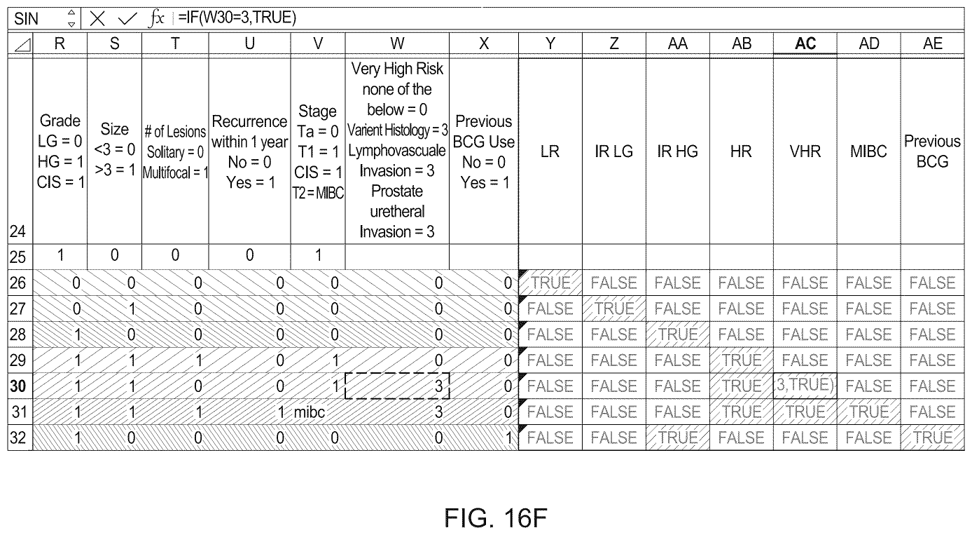

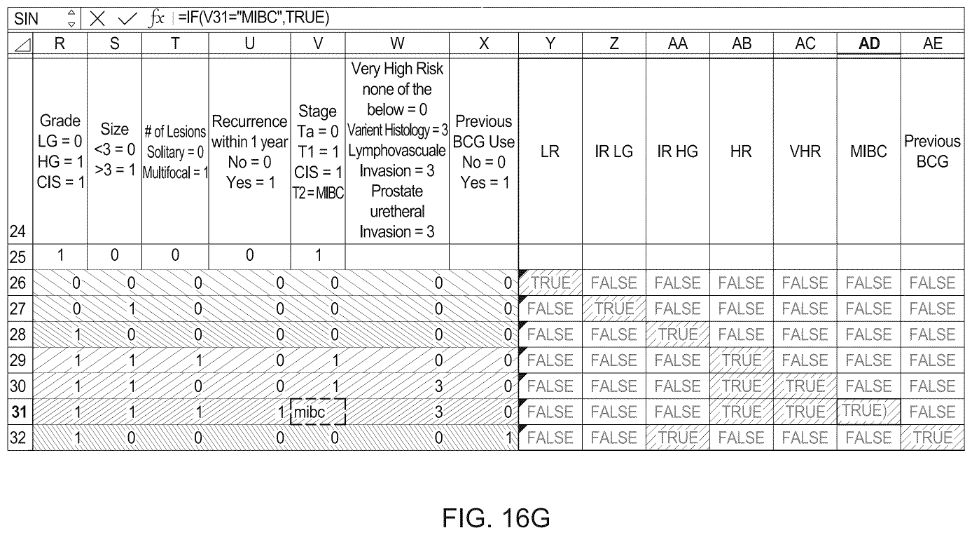

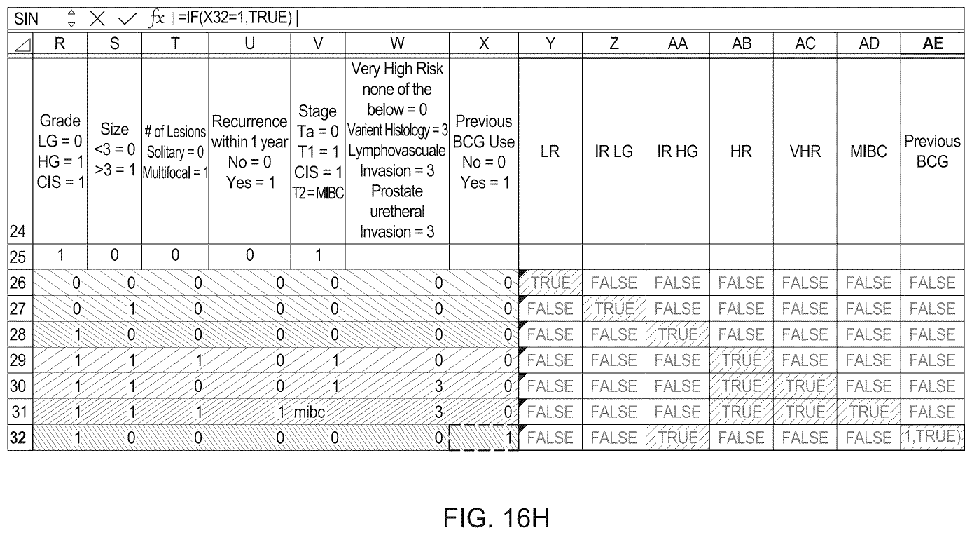

FIG. 16a-16h demonstrates how the semi-structured data inputted in FIG. 15 is used to determine an individual patient's non-muscle invasive risk group and to identify those men with muscle invasive disease. Columns “Y-AE” pull data previously entered in columns “R-X”. In this program the cell highlighted in red and labeled “TRUE” determines the patient risk group. In the situation that multiple cells in a row are red and labeled “TRUE” the column furthest to the right (column “Y-AD) is the actual risk group. It needs to be stated that previous BCG, column “AE” is not a risk group. To provide more detail each risk-groups formula is highlighted. Column “Y” is used to identify men with Low-Risk Non-muscle invasive bladder cancer. As shown in FIG. 16b, all data points in columns “R-W” have to be zero. If any column is greater than zero the patient cannot have low-risk bladder cancer. If all data points in “R-W” are zero the result of the formula is “TRUE”. A value of “FALSE” indicates the patient does not have low-risk non-muscle invasive bladder cancer. Column “Z” is used to identify men with intermediate-risk disease with low-grade histology. As shown in FIG. 16c, column “R” in the corresponding row must equal “0” or low-grade and at least one remaining column “S-V” has to be equal to one. If these criteria are met the results of the formula will be “TRUE”. Column “AA” is used to identify men with intermediate-risk disease with high-grade histology. As shown is FIG. 16d, column “R” in the corresponding row must equal “1” and the remaining values in columns “S-W” must be zero. If these criteria are meet the result of the formula will be “TRUE”. Column “AB” is used to identify men with high-risk disease. As shown in FIG. 16e, if column “R” in the corresponding row equals “1” and any of the values in columns “S, T, or V” equal “1” a value of “TRUE” is returned and the patient has at least high-risk non-muscle invasive bladder cancer. Column “AC” is used to identify men with very high-risk bladder cancer. As shown in FIG. 16f, if column “W” in the corresponding row equals 3 the patient has at least very high-risk non-muscle invasive bladder cancer. As shown in FIG. 16a a patient could have a TRUE statement for both high and very high-risk non-muscle invasive bladder cancer. When this occurs the TRUE statement further to the right (in this case very high-risk) is the correct risk group. Column “AD” is used to identify patients with muscle invasive bladder cancer (MIBC). As shown in FIG. 16g, if column “V” in the corresponding row equals “MIBC” the statement is “TRUE”. It is not infrequent that our program could show a patient to have a TRUE statement for High, Very High-Risk, and MIBC. In this scenario MIBC is furthest to the right and is the correct risk group. Column “AE” is used to identify patients with previous BCG use. As shown in FIG. 16h, if column “X” in the corresponding row equals “0” no previous BCG was given and if “1” previous BCG was given. It is important to note that previous BCG use is not a risk-group and does not apply to the previously statement of being further to the right.

While the exemplary embodiments illustrated in the figures and described herein are presently preferred, it should be understood that these embodiments are offered by way of example only. Accordingly, the present application is not limited to a particular embodiment, but extends to various modifications that nevertheless fall within the scope of the appended claims. The order or sequence of any processes or method steps may be varied or re-sequenced according to alternative embodiments.

It is important to note that the construction and arrangement of the various exemplary embodiments is illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited in the claims. For example, elements shown as integrally formed may be constructed of multiple parts or elements, the position of elements may be reversed or otherwise varied, and the nature or number of discrete elements or positions may be altered or varied. Accordingly, all such modifications are intended to be included within the scope of the present application. The order or sequence of any process or method steps may be varied or re-sequenced according to alternative embodiments. In the claims, any means-plus-function clause is intended to cover the structures described herein as performing the recited function and not only structural equivalents but also equivalent structures. Other substitutions, modifications, changes and omissions may be made in the design, operating conditions and arrangement of the exemplary embodiments without departing from the scope of the present application.

1. A patient management system comprising:
a risk analysis module configured to convert unstructured data to structured data, the risk analysis module including:
a data acquisition module for collecting and transmitting unstructured data;
a classifier module for analyzing unstructured data and converting the unstructured data into structured data;
wherein the data acquisition module receives an unstructured data set and the classifier module converts the unstructured data set to a structured data set;
a provider interface system in communication with the classifier module and configured to collect a plurality of structured data sets, analyze the collected structured data sets and output a risk profile to a provider.

2. The patient management system of claim 1, further comprising an analysis module to analyze the unstructured data set to provide the structured data set with the classifier module.

3. The patient management system of claim 1, wherein the risk profile corresponds to cancer risk groups and further diagnostic tests and therapeutics for prostate cancer.

4. The patient management system of claim 1, wherein the risk profile corresponds to cancer risk groups and further diagnostic tests and therapeutics for bladder cancer.

5. The patient management system of claim 1, wherein the provider further generates a care plan corresponding to the risk profile.

6. The patient management system of claim 1, further comprising a testing system for providing a diagnostic test for cancer, the testing system generating an unstructured data set for transfer to the risk analysis module.

7. A non-transitory machine-readable storage medium storing one or more sequences of instructions for generation of a risk profile for a cancer patient, which when executed by one or more processors, cause:
a risk analysis module in communication with the testing system to convert unstructured data to structure data, the risk analysis module including:
a data acquisition module to receive an unstructured data set;
a classifier module to analyze the unstructured data set and convert the unstructured data into a structured data set;
a provider interface system in communication with the classifier module to collect a plurality of structured data sets, analyze the collected structured data sets and output a risk profile to a provider.

8. A non-transitory machine-readable storage medium according to claim 7, further causing:
analysis of unstructured data with an analysis module to provide structured data with the classifier module.

9. A non-transitory machine-readable storage medium according to claim 7, further causing:
generation of a care plan corresponding to the risk profile.

10. A non-transitory machine-readable storage medium according to claim 7, wherein the risk profile corresponds to cancer risk groups and further diagnostic tests and therapeutics for prostate cancer.

11. A non-transitory machine-readable storage medium according to claim 7, wherein the risk profile corresponds to cancer risk groups and further diagnostic tests and therapeutics for bladder cancer.

12. A non-transitory machine-readable storage medium according to claim 7, further comprising a testing system for providing a diagnostic test for cancer, the testing system generating an unstructured data set for transfer to the risk analysis module.

13. A method for providing a risk profile and care plan to a patient, the method comprising:
providing an unstructured data set;
converting unstructured data set to a structured data set with a risk analysis module in communication with the testing system;
aggregating a plurality of structured data sets with a provider interface system to determine a risk score; and
providing the risk score to a provider with the provider interface system.

14. The method of claim 13, wherein the risk analysis module comprises:
a data acquisition module for collecting and transmitting the unstructured data set; and
a classifier module for analyzing the unstructured data set and converting the unstructured data set into the structured data set;
wherein the data acquisition module receives the unstructured data set from the testing system and the classifier module converts the unstructured data to a structured data set.

15. The method of claim 13, further comprising analyzing unstructured data with an analysis module to provide structured data with the classifier module.

16. The patient management system of claim 13, wherein the risk profile corresponds to cancer risk groups and further diagnostic tests and therapeutics for prostate cancer.

17. The method of claim 13, wherein the risk profile corresponds to cancer risk groups and further diagnostic tests and therapeutics for bladder cancer.

18. The method of claim 13, further comprising providing a patient with a care plan corresponding to the risk score.

19. The method of claim 13, further providing a diagnostic test for cancer with a testing system, the testing system generating an unstructured data set for transfer to the risk analysis module.
